(19) United States
(12) Patent Application Publication
(10) Pub. No.: US 2024/0091323 A1
(43) Pub. Date: Mar. 21, 2024

(54) ENGINEERED STABLE ACE2 PROTEIN VARIANTS AS ANTIVIRAL NEBULIZED THERAPEUTICS

(71) Applicant: THE BOARD OF REGENTS OF THE UNIVERSITY OF TEXAS SYSTEM, Austin, TX (US)

(72) Inventors: Daniel M. Rosenbaum, Dallas, TX (US); Daniel Kober, Dallas, TX (US)

(73) Assignee: THE BOARD OF REGENTS OF THE UNIVERSITY OF TEXAS SYSTEM, Austin, TX (US)

(21) Appl. No.: 18/271,173

(22) PCT Filed: Jan. 7, 2022
(86) PCT No.: PCT/US2022/011685

§ 371 Date: Jul. 6, 2023

Related U.S. Application Data
(60) Provisional application No. 63/134,751, filed on Jan. 7, 2021.

(51) Int. Cl. A61K 38/48; A61K 31/573; A61K 31/675; A61K 47/60; A61P 31/14

(52) U.S. Cl. CPC A61K 38/4813; A61K 31/573; A61K 31/675; A61K 47/60; A61P 31/14; C12Y 304/17023

(57) ABSTRACT

Disclosed herein are ACE2 recombinant proteins and methods of using such for therapeutic and/or diagnostic purposes. Also provided herein are methods for producing such recombinant proteins.

### CROSS REFERENCE TO RELATED APPLICATIONS

This application is a national stage application filed under 35 U.S.C. § 371 of International Patent Application No. PCT/US2022/11685 entitled “ENGINEERED STABLE ACE2 PROTEIN VARIANTS AS ANTIVIRAL NEBULIZED THERAPEUTICS” and filed on Jan. 7, 2022, which claims priority to U.S. Provisional Application No. 63/134,751 entitled “ENGINEERED STABLE ACE2 PROTEIN VARIANTS AS ANTIVIRAL NEBULIZED THERAPEUTICS” and filed Jan. 7, 2021, the contents of each are hereby incorporated by reference in their entireties.

### STATEMENT REGARDING SEQUENCE LISTING

An electronic version of the Sequence Listing is filed herewith, the contents of which are incorporated by reference in their entirety. The electronic file is 109 kilobytes in size, and titled WO_UTSD3793_SequenceListing_ST25.txt.

### BACKGROUND

### 1. Field

The present inventive concept is directed to compositions and methods of administering ACE2 recombinant proteins with improved stability and methods of using such for therapeutic and/or diagnostic purposes.

### 2. Discussion of Related Art

A novel coronavirus, first reported in Wuhan City, China, was later named severe acute respiratory syndrome coronavirus 2 (SARS-CoV-2). SARS-CoV-2 is a RNA virus classified as the seventh subtype of the coronavirus family. Four of the six coronavirus subtypes are less pathogenic than SARS-CoV-2 and usually result in mild catarrhal inflammation after infection; however, two previously identified coronavirus subtypes, known as the viruses causing SARS-CoV and Middle East Respiratory Syndrome (MERS), have rapid transmission rates. Alarmingly, SARS-CoV-2 spreads more efficiently than SARS-CoV in 2003 and MERS-CoV in 2015. After infection, SARS-CoV-2 causes coronavirus disease 2019 (COVID-19). Infection with SARS-CoV-2 results in atypical pneumonia with symptoms including fever, coughing, fatigue, and breathing difficulties. To date, there are few if any therapies for the treatment of SARS-CoV-2 infection. As such, there is a need to develop new therapies for the treatment of SARS-CoV-2 infection as well as respiratory infection(s) resulting from any virus that uses ACE2 as a co-receptor.

Biopharmaceutical delivery by inhalation is a delivery route which offers high potential for direct local lung application of a biopharmaceutical for treatment of respiratory infections, including a SARS-CoV-2 infection. Nebulizers are generally a preferred choice for atomization of liquid biopharmaceutical formulations as nebulization can avoid additional process steps needed for other inhaled delivery forms. Although small molecules can survive the stresses encountered during nebulization, protein-based biopharmaceuticals usually lack the stability to remain stable throughout aerosolization. Accordingly, there is a need to develop a highly stable, protein-based therapy for respiratory infections resulting from a virus that uses ACE2 as a co-receptor (e.g., a SARS-CoV-2 infection) suitable for delivery by nebulization.

### SUMMARY OF THE INVENTION

The present disclosure is based, at least in part, on the development of a recombinant human ACE protein with improved stability throughout aerosolization. An aspect of the present disclosure provides a recombinant protein, comprising a recombinant human ACE protein fragment of an angiotensin-converting enzyme 2 (ACE2) receptor. In some embodiments, the fragment disclosed herein can have at least 80% identity to an amino acid sequence represented by SEQ ID NOs: 3-9 or a nucleic acid encoding the fragment having at least 80% identity with the polynucleotide represented by SEQ ID NOs: 11-17. In some embodiments, a recombinant human ACE protein fragment herein can have at least one binding site for a spike protein of a coronavirus. In some embodiments, the coronavirus can be SARS-CoV-2.

In some aspects, a recombinant ACE protein herein can be an ACE2 receptor. In some embodiments, a recombinant ACE protein herein can be a human ACE2 receptor. In some embodiments, a recombinant ACE protein herein can be a fragment of the ACE2 receptor. In some embodiments, a fragment of the ACE2 receptor can include the ectodomain of the ACE2 receptor. In some embodiments, a recombinant ACE protein herein can further include a signaling peptide at the N-terminus.

In some embodiments, a recombinant ACE protein herein can have at least 90% identity to the amino acid sequence represented by SEQ ID NOs: 3-9. In some other embodiments, a recombinant ACE protein herein can have a polynucleotide sequence encoding a fragment of an ACE2 receptor having at least 90% identity to SEQ ID NOs: 11-17. In an exemplary embodiment, a recombinant ACE protein herein has an amino acid sequence of SEQ ID NO: 10. In another exemplary embodiment, a recombinant ACE protein herein has a polynucleotide sequence of SEQ ID NO: 18.

In another aspect, the present disclosure provides pharmaceutical compositions that can include at least one of the recombinant ACE proteins disclosed herein and a pharmaceutically acceptable carrier. In some embodiments, pharmaceutical compositions disclosed herein can be a nebulized solution.

In still another aspect, the present disclosure provides methods of treating a coronavirus infection, inhibiting a coronavirus infection, or any combination thereof. In some embodiments, methods herein can include administering to a subject in need thereof an effective amount of at least one of the recombinant ACE proteins disclosed herein and/or at least one of the pharmaceutical compositions disclosed herein. In some embodiments, pharmaceutical compositions disclosed herein can be administered by a nebulizer. In some embodiments, a subject in need thereof can be a human subject. In some embodiments, a human subject in need thereof can be suspected of having, or at risk for the coronavirus infection. In some embodiments, a coronavirus infection can be a SARS-CoV-2 infection. In some other embodiments, a subject in need thereof can be a human patient having or suspected of having COVID-19.

In some embodiments, methods of administering recombinant ACE proteins disclosed herein to a subject in need thereof can further include administration of an effective amount of least one anti-viral agent to the subject. In some embodiments, the at least one anti-viral agent can be remdesivir. In some embodiments, methods of administering recombinant ACE proteins disclosed herein to a subject in need thereof can further include administration of an effective amount of at least one corticosteroid to the subject. In some embodiments, the at least one corticosteroid can be dexamethasone.

Additional aspects, advantages, and utilities of the present inventive concept are set forth, in part, in the description which follows. The details of one or more embodiments of the inventive concepts disclosed herein are set forth in the description below. Other features or advantages of the present disclosure are apparent from the following drawings and detailed description of several embodiments, and also from the appended claims.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

Embodiments of the present inventive concept are illustrated by way of example in which like reference numerals indicate similar elements and in which:

FIG. 1 depicts an image illustrating the key residues identified by examining the inhibitor-bound ACE2 enzymatic domain (PDB 1R4L). The bound inhibitor (MLN-4760) and side chains of the identified residues are shown as sticks.

FIGS. 2A-2C depict images illustrating the design and expression of secreted ACE2 ectodomains as provided herein. FIG. 2/1 shows a schematic for the ACE2 ectodomain construct where, beginning from the amine terminus, the construct encoded the secretion signal from Honeybee Melittin, human ACE2 ectodomain residues 19-740, a TEV recognition site (ENLYFQG), a TG spacer, and 10 His residues. The ACE2 ectodomain was subdivided into its enzymatic domain (residues 19-615) and its neck domain (residues 616-740). FIG. 2B shows representative images of Western blots depicting expression of the ACE2 ectodomains harvested from supernatants of Sf9 cells that were uninfected (−) or infected with baculovirus for either ACE2 wild-type (WT) or ACE2 T371W (T371W). Supernatants were immunoblotted with anti-His or anti-ACE2 antibodies to detect secreted ACE2 ectodomains. FIG. 2C shows the design of the T371W mutation that prevented ACE2 protease activity using a crystal structure of the inhibitor-bound ACE2 enzymatic domain (PDB 1R4L) was used to identify residues which could be mutated to fill the binding pocket and prevent substrate engagement. Residue T371 projects into the binding pocket. The T371W mutation inserts a large sidechain into the binding pocket.

FIGS. 3A-3D depict images illustrating the purification and characterization of secreted ACE2 ectodomains. FIG. 3A shows a graph depicting the gel filtration analysis of purified ACE2 ectodomains. FIG. 3B shows a representative image of a SDS-PAGE analysis of purified ACE2 ectodomains. FIG. 3C shows a graph depicting the oligomeric status of the ACE2 T371W as determined by Size Exclusion Chromatography Multi-Angle Light Scattering (SEC-MALS) where the ACE2 ectodomain eluted as a single peak, the weight of the weight of carbohydrates was determined and, resulting average molar mass of the protein component was determined to be 163,000±1,000 g/mol, consistent with a dimeric species. FIG. 3D shows a representative image of a SDS-PAGE analysis of an ACE2 T371W ectodomain treated buffer (−) or with PNGaseF (+) where treatment with PNGaseF resulted in a protein that migrated as a smaller species (indicated on the gel by * asterisk), revealing the presence of N-linked glycans.

FIGS. 4A-4C depict graphs illustrating the stability of a mutant ACE2 ectodomain. FIG. 4A shows a graph depicting a differential scanning fluorimetry fluorescence analysis of ACE2 T371W before and after removal of glycans by PNGaseF treatment. FIG. 4B shows a graph depicting the derivative analysis of the traces shown in FIG. 4A. FIG. 4C shows a graph depicting a Circular Dichroism melt analysis of ACE2 T371W.

FIGS. 5A and 5B depict images illustrating the binding affinity of a mutant ACE2 ectodomain to SARS2-RBD. FIG. 5A shows a graph depicting the binding affinity for the ACE2 T371W and the SARS2-RBD (SARS-CoV-2 Spike receptor binding domain) as determined by Isothermal Titration calorimetry (ITC) where the binding was determining to be a 1:1 interaction between the RBD and ACE2 monomers with a binding constant of 15.1 nM (95% confidence interval of 8-24 nM) and the change in enthalpy (ΔH) was determined to be −15.7 kcal/mol (95% confidence interval −15.9-−15.4 kcal/mol). FIG. 5B shows a representative image of a SDS-PAGE analysis depicting the purity of the protein samples used in the analysis shown in FIG. 5A.

FIGS. 6A-6C depict images illustrating the tolerability of a mutant ACE2 ectodomain to nebulization. FIG. 6A shows a graph depicting the elution peak of purified ACE2 T371W as analyzed by Size Exclusion Chromatography over a Superdex S200 10/300 column. FIG. 6B shows a graph depicting the elution peak of purified ACE2 T371W after the ectodomain was aerosolized by vibrating mesh using a NEB400 Mini Mesh Nebulizer as analyzed by Size Exclusion Chromatography over a Superdex S200 10/300 column. The elution pattern revealed the nebulized ACE2 T371W to be free of aggregates and to elute as a homodimer at the same volume as the native ACE2 protein. FIG. 6C shows a representative image of a SDS-PAGE analysis depicting the ACE2 T371W sample before and after nebulization.

FIGS. 7A and 7B depict graphs illustrating effectiveness of a solubilized mutant ACE2 ectodomain and a nebulized-mutant ACE2 ectodomain at inhibition of viral replication of SARS-CoV-2. FIG. 7A shows a graph depicting the percentage of infected cells with increasing concentrations of solubilized mutant ACE2 ectodomain and nebulized-mutant ACE2 ectodomain. FIG. 7B shows a graph depicting the percentage of infectivity (% of control) with increasing concentrations of solubilized mutant ACE2 ectodomain and nebulized-mutant ACE2 ectodomain. FIGS. 7A and 7B show that both the solubilized ACE2 T371W and the nebulized ACE2 T371W had an EC₅₀ of about 1-2 nM.

FIGS. 8A and 8B depict graphs illustrating effectiveness of a stabilized mutant ACE2 ectodomain at treating SARS-CoV-2 in a mouse model. FIG. 8A shows a graph depicting daily weight loss in mice for up to nine days after intranasal infection with SARS-CoV-2, wherein the mice were either untreated, pre-treated, or treated post-infection with a stabilized mutant ACE2 administered intranasally as indicated. FIG. 8B shows a Kaplan-Meier survival curve depicting survival probability in SARS-CoV-2-infected mice that were either untreated, pre-treated, or treated post-infection with a stabilized mutant ACE2 administered intranasally as indicated.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the development of a recombinant protein capable of blocking entry of coronavirus into host cells via ACE2. Such a recombinant protein can include ACE2 receptor or a fragment thereof capable of binding to a Spike protein of a coronavirus (e.g., the Spike protein of SARS-CoV2). In certain embodiments, recombinant proteins disclosed herein can prevent a coronavirus from fusing with the cell membrane by specifically binding the Spike protein with high affinity. In certain embodiments, recombinant proteins disclosed herein can include specific mutations to the endogenous ACE2 amino acid sequence that improves stability of the protein when compared to unmutated ACE2 receptor proteins. In certain embodiments, recombinant proteins disclosed herein can include an ACE2 receptor or a fragment thereof capable of retaining protein stability during throughout aerosolization. In certain embodiments, recombinant proteins disclosed herein can include specific mutations to the endogenous ACE2 amino acid sequence that improve pharmacokinetic properties and bioavailability compared to unmutated ACE2 receptor proteins and have the ability to be nebulized in a therapeutic context.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein a “recombinant protein” refers to any protein and/or polypeptide that can be administered to a mammal to elicit a biological or medical response of a tissue, system, animal or human. A recombinant protein can elicit more than one biological or medical response. Furthermore, the term “therapeutically effective amount” refers to any amount which, as compared to a corresponding subject who has not received such amount, results in, but is not limited to, healing, prevention, or amelioration of a disease, disorder, or side effect, or a decrease in the rate of advancement of a disease or disorder. The term also includes within its scope amounts effective to enhance normal physiological function as well as amounts effective to cause a physiological function in a subject which enhances or aids in the therapeutic effect of a second pharmaceutical agent and/or treatment regimen.

As used herein “a nebulizer” refers to a device which is capable of aerosolizing a liquid material into a dispersed liquid phase. As used herein, “an aerosol” refers to a system comprising a continuous gas phase and, dispersed therein, a discontinuous or dispersed phase of liquid particles.

### II. Compositions

### (a) ACE2 Recombinant Proteins

In some aspects, the present disclosure provides recombinant proteins capable of binding to a spike protein of a coronavirus, such as the SARS-CoV-2 spike protein “5”. Recombinant proteins, such as those described herein, have a higher affinity and/or abundance for the viral spike protein then the native ACE2 receptor of the virus. As such, the recombinant proteins disclosed herein can reduce or prevent a virus (e.g., SARS-CoV-2) via its Spike protein from binding to the ACE2 receptors on host cells for infection. In certain embodiments, recombinant proteins disclosed herein may be used for either therapeutic or diagnostic purposes to prevent, treat or diagnose an infection caused by a virus (e.g., a coronavirus such as SARS-CoV-2). In some embodiments, recombinant proteins disclosed herein can be used for treating COVID-19.

In some embodiments, recombinant proteins disclosed herein can be capable of binding to the to the S1 subunit of SARS-CoV-2 spike protein S. In some embodiments, recombinant proteins disclosed herein can be capable of binding to the receptor binding domain (RBD). In some embodiments, recombinant proteins disclosed herein can be capable of binding to the SARS-CoV-2 RBD with a binding affinity (K_{D}) of about 10 nM to about 20 nM. In some preferred embodiments, recombinant proteins disclosed herein can be capable of binding to the SARS-CoV-2 RBD with a binding affinity (K_{D}) of about 15 nM. In some embodiments, K_{D} values for ACE2 recombinant proteins described herein can remain unchanged following nebulization (e.g., aerosolization) of the ACE2 recombinant protein.

Any of the ACE2 recombinant proteins described herein can inhibit (e.g., reduce or eliminate) the ability of a virus (e.g., SARS-CoV-2) to enter into host cells and undergo viral replication therein. In some embodiments, ACE2 recombinant proteins as described herein can inhibit SARS-CoV-2 replication by at least about 30% (e.g., about 35%, about 40%, about 45%, about 50%, about 60%, about 70%, about 80%, about 90%, about 95% or greater, including any increment therein). In some embodiments, ACE2 recombinant proteins described herein can inhibit viral (e.g., SARS-CoV-2) replication following nebulization (e.g., aerosolization) with the same effectiveness as ACE2 recombinant proteins described herein that are not nebulized. The inhibitory activity of ACE2 recombinant proteins on SARS-CoV-2 replication described herein can be determined by routine methods known in the art, e.g., by an assay for measuring the percentage inhibition of virus yield.

In certain embodiments, ACE2 recombinant proteins described herein can have a virus-inhibitory effect with a 50% effective concentration (EC₅₀) ranging from about 0.5 nM to about 5 nM. In some embodiments, ACE2 recombinant proteins described herein can have a virus-inhibitory effect with an EC₅₀ ranging from about 1 to about 2 nM. In some embodiments, ACE2 recombinant proteins described herein can have a SARS-CoV-2-inhibitory effect with an EC₅₀ ranging from about 0.5 nM to about 5 nM. In some preferred embodiments, ACE2 recombinant proteins described herein can have a SARS-CoV-2-inhibitory effect with an EC₅₀ ranging from about 1 nM to about 2 nM. In some embodiments, EC50 values for ACE2 recombinant proteins described herein can remain unchanged following nebulization (e.g., aerosolization) of the ACE2 recombinant protein.

In some embodiments, the percent inhibition of virus yield by an ACE recombinant protein may be calculated as:

[1−(Vd/Vc)]×100%

where Vd and Vc refer to the virus copies in the in the presence and absence of the test compound. In some embodiments, any of the recombinant proteins as described herein, e.g., the solubilized and/or nebulized ACE2 recombinant proteins provided herein, can result in about a 30%, about a 35%, about a 40%, about a 45%, about a 50%, about a 55%, about a 60%, about a 65%, about a 70%, about a 75%, about a 80%, about a 85%, about a 90%, about a 95%, about a 99%, or greater percentage inhibition of SARS-CoV-2 virus yield.

The present disclosure provides for ACE2 recombinant proteins having at least one binding site of an ACE2 receptor for a spike protein of a coronavirus. In some embodiments, an ACE2 recombinant protein herein may encompass a full length human ACE2 (SEQ ID NO: 1). In some embodiments, an ACE2 recombinant protein herein may encompass a fragment of human ACE2 that contains at least one binding site to a spike protein of a coronavirus. In certain embodiments, a fragment of ACE2 may include the complete ectodomain (SEQ ID NO: 2), a portion of the ectodomain, the complete transmembrane domain, a portion of the transmembrane domain, the complete cytoplasmic tail, a portion of the cytoplasmic tail, or any combination thereof.

In some embodiments, an ACE2 recombinant protein disclosed herein may encompass an ACE2 ectodomain domain or a fragment thereof comprising at least one binding site to a spike protein of a coronavirus. In some embodiments, an ACE2 recombinant protein herein may encompass at least one binding site for the subunit S1 of the spike protein of a coronavirus. In some embodiments, an ACE2 recombinant protein herein may encompass at least one binding site for receptor binding domain (RBD) of the spike protein of a coronavirus. In some embodiments, an ACE2 recombinant may include an ectodomain domain of an ACE2 and/or one of its active fragments.

In some embodiments, an ACE2 recombinant protein disclosed herein may encompass at least one mutation in its amino acid sequence. As used herein, a mutation to a protein amino acid sequence can result in expression of a protein having a different amino acid sequence compared to the endogenous amino acid sequence. In some embodiments, at least one amino acid residue to at least 20 amino acid residues can be mutated in an ACE2 recombinant protein disclosed herein as compared to the endogenous amino acid sequence. In some embodiments, at least about 1, about 2, about 3, about 4, about 5, about 6, about 7, about 8, about 9, about 10, about 11, about 12, about 13, about 14, about 15, about 16, about 17, about 18, about 19, or about 20 amino acid residues can be mutated in an ACE2 recombinant protein disclosed herein as compared to the endogenous amino acid sequence.

In some embodiments, a mutation in the amino acid sequence of in an ACE2 recombinant protein disclosed herein can increase the stability of the resulting ACE2 recombinant protein compared to an unmutated ACE2 recombinant protein. In some embodiments, a mutation in the amino acid sequence of in an ACE2 recombinant protein disclosed herein can increase the thermostability of the resulting ACE2 recombinant protein compared to an unmutated ACE2 recombinant protein. In some embodiments, a mutation in the amino acid sequence of in an ACE2 recombinant protein disclosed herein can increase the binding affinity of the resulting ACE2 recombinant protein for the spike protein of a coronavirus compared to an unmutated ACE2 recombinant protein. In some embodiments, a mutation in the amino acid sequence of in an ACE2 recombinant protein disclosed herein can increase the bioavailability of the resulting ACE2 recombinant protein for the spike protein of a coronavirus compared to an unmutated ACE2 recombinant protein. In some embodiments, a mutation in the amino acid sequence of in an ACE2 recombinant protein disclosed herein can increase the stability of the resulting ACE2 recombinant protein following aerosolization compared to an unmutated ACE2 recombinant protein following aerosolization. In some embodiments, a mutation in the amino acid sequence of in an ACE2 recombinant protein disclosed herein can increase the stability of the resulting ACE2 recombinant protein for nebulization compared to an unmutated ACE2 recombinant protein.

In some embodiments, ACE2 recombinant proteins disclosed herein may have an ACE polypeptide having a sequence of any of the proteins in Table 1 below.

TABLE 1

| Protein/ | | SEQ |
|---|---|---|
| Construct | | ID |
| Name | Amino Acid Sequence | NO: |
| WT ACE2 | MKFLVNVALVEMVVYISYIYASTIEEQAKTFLDKENHEAEDLFYQSSL | 3 |
| | ASWNYNTNITEENVQNMNNAGDKWSAFLKEQSTLAQMYPLQEIQNLTV | |
| | KLQLQALQQNGSSVLSEDKSKRLNTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGREWTNLYSLTVPF | |
| | GQKPNIDVTDAMVDQAWDAQRIFKEAEKFFVSVGLPNMTQGFWENSML | |
| | TDPGNVQKAVCHPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVEKGEIPKDQWMKKW | |
| | WEMKREIVGVVEPVPHDETYCDPASLFHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLFRSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH | |
| ACE2 T371X | MKFLVNVALVEMVVYISYIYASTIEEQAKTFLDKENHEAEDLFYQSSL | 4 |
| | ASWNYNTNITEENVQNMNNAGDKWSAFLKEQSTLAQMYPLQEIQNLTV | |
| | KLQLQALQQNGSSVLSEDKSKRLNTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGREWTNLYSLTVPF | |
| | GQKPNIDVTDAMVDQAWDAQRIFKEAEKFFVSVGLPNMTQGFWENSML | |
| | TDPGNVQKAVCHPTAWDLGKGDFRILMCTKVTMDDFLXAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVFKGEIPKDQWMKKW | |
| | WEMKREIVGVVEPVPHDETYCDPASLFHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLFRSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH | |
| N149X ACE2 | MKFLVNVALVEMVVYISYIYASTIEEQAKTFLDKENHEAEDLFYQSSL | 5 |
| | ASWNYNTNITEENVQNMNNAGDKWSAFLKEQSTLAQMYPLQEIQNLTV | |
| | KLQLQALQQNGSSVLSEDKSKRLNTILNTMSTIYSTGKVCNPDNPQEC | |
| | LLLEPGLXEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGREWTNLYSLTVPF | |
| | GQKPNIDVTDAMVDQAWDAQRIFKEAEKFFVSVGLPNMTQGFWENSML | |
| | TDPGNVQKAVCHPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVEKGEIPKDQWMKKW | |
| | WEMKREIVGVVEPVPHDETYCDPASLFHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLERSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH* | |
| F274X ACE2 | MKFLVNVALVEMVVYISYIYASTIEEQAKTFLDKENHEAEDLFYQSSL | 6 |
| | ASWNYNTNITEENVQNMNNAGDKWSAFLKEQSTLAQMYPLQEIQNLTV | |
| | KLQLQALQQNGSSVLSEDKSKRINTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGRXWTNLYSLTVPE | |
| | GQKPNIDVTDAMVDQAWDAQRIFKEAEKFFVSVGLPNMTQGFWENSML | |
| | TDPGNVQKAVCHPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVFKGEIPKDQWMKKW | |
| | WEMKREIVGVVEPVPHDETYCDPASLFHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLERSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH* | |
| H345X ACE2 | MKFLVNVALVEMVVYISYIYASTIEEQAKTELDKENHEAEDLFYQSSL | 7 |
| | ASWNYNTNITEENVQNMNNAGDKWSAFLKEQSTLAQMYPLQEIQNLTV | |
| | KLQLQALQQNGSSVLSEDKSKRINTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGREWTNLYSLTVPF | |
| | GQKPNIDVTDAMVDQAWDAQRIFKEAEKFFVSVGLPNMTQGFWENSML | |
| | TDPGNVQKAVCXPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVFKGEIPKDQWMKKW | |
| | WEMKREIVGVVEPVPHDETYCDPASLFHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLERSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH* | |
| F504X ACE2 | MKFLVNVALVEMVVYISYIYASTIEEQAKTFLDKENHEAEDLFYQSSL | 8 |
| | ASWNYNTNITEENVQNMNNAGDKWSAFLKEQSTLAQMYPLQEIQNLTV | |
| | KLQLQALQQNGSSVLSEDKSKRINTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGRFWTNLYSLTVPE | |
| | GQKPNIDVTDAMVDQAWDAQRIFKEAEKFFVSVGLPNMTQGEWENSML | |
| | TDPGNVQKAVCHPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVEKGEIPKDQWMKKW | |
| | WEMKREIVGVVEPVPHDETYCDPASLXHVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLERSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH* | |
| H505X ACE2 | MKFLVNVALVEMVVYISYIYASTIEEQAKTFLDKENHEAEDLFYQSSL | 9 |
| | ASWNYNTNITEENVQNMNNAGDKWSAFLKEQSTLAQMYPLQEIQNLTV | |
| | KLQLQALQQNGSSVLSEDKSKRINTILNTMSTIYSTGKVCNPDNPQEC | |
| | LLLEPGLNEIMANSLDYNERLWAWESWRSEVGKQLRPLYEEYVVLKNE | |
| | MARANHYEDYGDYWRGDYEVNGVDGYDYSRGQLIEDVEHTFEEIKPLY | |
| | EHLHAYVRAKLMNAYPSYISPIGCLPAHLLGDMWGREWTNLYSLTVPF | |
| | GQKPNIDVTDAMVDQAWDAQRIFKEAEKFFVSVGLPNMTQGFWENSML | |
| | TDPGNVQKAVCHPTAWDLGKGDFRILMCTKVTMDDELTAHHEMGHIQY | |
| | DMAYAAQPFLLRNGANEGFHEAVGEIMSLSAATPKHLKSIGLLSPDFQ | |
| | EDNETEINFLLKQALTIVGTLPFTYMLEKWRWMVFKGEIPKDQWMKKW | |
| | WEMKREIVGVVEPVPHDETYCDPASLEXVSNDYSFIRYYTRTLYQFQF | |
| | QEALCQAAKHEGPLHKCDISNSTEAGQKLENMLRLGKSEPWTLALENV | |
| | VGAKNMNVRPLLNYFEPLFTWLKDQNKNSFVGWSTDWSPYADQSIKVR | |
| | ISLKSALGDKAYEWNDNEMYLFRSSVAYAMRQYFLKVKNQMILFGEED | |
| | VRVANLKPRISENFFVTAPKNVSDIIPRTEVEKAIRMSRSRINDAFRL | |
| | NDNSLEFLGIQPTLGPPNQPPVSENLYFQGTGHHHHHHHHHH* | |
| Full length | MSSSSWLLLSLVAVTAAQSTIEEQAKTELDKENHEAEDLFYQSSLASW | 1 |
| Human ACE2 | NYNTNITEENVQNMNNAGDKWSAFLKEQSTLAQMYPLQEIQNLTVKLQ | |
| | LQALQQNGSSVLSEDKSKRLNTILNTMSTIYSTGKVCNPDNPQECLLL | |
| | EPGLNEIMANSLDYNERLWAWESWRSEVGKQLRPLYEEYVVLKNEMAR | |
| | ANHYEDYGDYWRGDYEVNGVDGYDYSRGQLIEDVEHTFEEIKPLYEHL | |
| | HAYVRAKLMNAYPSYISPIGCLPAHLLGDMWGREWTNLYSLTVPFGQK | |
| | PNIDVTDAMVDQAWDAQRIFKEAEKFFVSVGLPNMTQGFWENSMLTDP | |
| | GNVQKAVCHPTAWDLGKGDFRILMCTKVTMDDFLTAHHEMGHIQYDMA | |
| | YAAQPFLLRNGANEGFHEAVGEIMSLSAATPKHLKSIGLLSPDFQEDN | |
| | ETEINFLLKQALTIVGTLPFTYMLEKWRWMVFKGEIPKDQWMKKWWEM | |
| | KREIVGVVEPVPHDETYCDPASLFHVSNDYSFIRYYTRTLYQFQFQEA | |
| | LCQAAKHEGPLHKCDISNSTEAGQKLENMLRLGKSEPWTLALENVVGA | |
| | KNMNVRPLLNYFEPLFTWLKDQNKNSFVGWSTDWSPYADQSIKVRISL | |
| | KSALGDKAYEWNDNEMYLERSSVAYAMRQYFLKVKNQMILFGEEDVRV | |
| | ANLKPRISENFFVTAPKNVSDIIPRTEVEKAIRMSRSRINDAFRLNDN | |
| | SLEFLGIQPTLGPPNQPPVSIWLIVFGVVMGVIVVGIVILIFTGIRDR | |
| | KKKNKARSGENPYASIDISKGENNPGFQNTDDVQTSF | |
| Ectodomain of | QSTIEEQAKTELDKENHEAEDLFYQSSLASWNYNTNITEENVQNMNNA | 2 |
| human ACE2 | GDKWSAFLKEQSTLAQMYPLQEIQNLTVKLQLQALQQNGSSVLSEDKS | |
| | KRLNTILNTMSTIYSTGKVCNPDNPQECLLLEPGLNEIMANSLDYNER | |
| | LWAWESWRSEVGKQLRPLYEEYVVLKNEMARANHYEDYGDYWRGDYEV | |
| | NGVDGYDYSRGQLIEDVEHTFEEIKPLYEHLHAYVRAKLMNAYPSYIS | |
| | PIGCLPAHLLGDMWGRFWTNLYSLTVPFGQKPNIDVTDAMVDQAWDAQ | |
| | RIFKEAEKFFVSVGLPNMTQGFWENSMLTDPGNVQKAVCHPTAWDLGK | |
| | GDFRILMCTKVTMDDELTAHHEMGHIQYDMAYAAQPELLRNGANEGFH | |
| | EAVGEIMSLSAATPKHLKSIGLLSPDFQEDNETEINFLLKQALTIVGT | |
| | LPFTYMLEKWRWMVFKGEIPKDQWMKKWWEMKREIVGVVEPVPHDETY | |
| | CDPASLFHVSNDYSFIRYYTRTLYQFQFQEALCQAAKHEGPLHKCDIS | |
| | NSTEAGQKLENMLRLGKSEPWTLALENVVGAKNMNVRPLLNYFEPLFT | |
| | WLKDQNKNSFVGWSTDWSPYAD | |

In Table 1 above, the melittin signal peptide is in bold (MKFLVNVALVFMVVYISYIYA; SEQ ID NO: 19); the TEV recognition site is italicized (ENLYFQG; SEQ ID NO: 20); the TG spacer is italicized and underlined; the 10 His tag is underlined (HHHHHHHHHH; SEQ ID NO: 21); and amino acid residue mutations “X” are in bold and italicized.

In some embodiments, an ACE2 recombinant protein disclosed herein may have an ACE polypeptide sequence having a sequence of any of the polypeptides in Table wherein “X” denotes the site of a possible mutation for the polypeptide sequence. In some embodiments, a possible mutation(s) to any of the polypeptide sequences in Table at “X” can be: F, L, I, M, V, S, P, T, A, Y, H, Q, N, K, D, E, C, W, R, or G.

In some preferred embodiments, ACE2 recombinant proteins disclosed herein may comprise an ACE polypeptide sequence as follows:

ACE2 T371W:
(SEQ ID NO: 10)
MKFLVNVALVFMVVYISYIYASTIEEQAKTFLDKENHEAEDLFYQSSLAS
WNYNTNITEENVQNMNNAGDKWSAFLKEQSTLAQMYPLQEIQNLTVKLQL
QALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHYE
DYGDYWRGDYEVNGVDGYDYSRGQLIEDVEHTFEEIKPLYEHLHAYVRAK
LMNAYPSYISPIGCLPAHLLGDMWGRFWINLYSLTVPFGQKPNIDVTDAM
VDQAWDAQRIFKEAEKFFVSVGLPNMTQGFWENSMLTDPGNVQKAVCHPT
AWDLGKGDFRILMCTKVTMDDELWAHHEMGHIQYDMAYAAQPFLLRNGAN
EGFHEAVGEIMSLSAATPKHLKSIGLLSPDFQEDNETEINFLLKQALTIV
GTLPFTYMLEKWRWMVFKGEIPKDQWMKKWWEMKREIVGVVEPVPHDETY
CDPASLFHVSNDYSFIRYYTRTLYQFQFQEALCQAAKHEGPLHKCDISNS
TEAGQKLFNMLRLGKSEPWTLALENVVGAKNMNVRPLLNYFEPLFTWLKD
QNKNSFVGWSTDWSPYADQSIKVRISLKSALGDKAYEWNDNEMYLFRSSV
AYAMRQYFLKVKNQMILFGEEDVRVANLKPRISENFFVTAPKNVSDIIPR
TEVEKAIRMSRSRINDAFRLNDNSLEFLGIQPTLGPPNQPPVSENLYFQG
TGHHHHHHHHHH,

where the melittin signal peptide is in bold; the TEV recognition site is italicized; the TG spacer is italicized and underlined; the 10 His tag is underlined; and amino acid residue is in bold and italicized.

In some embodiments, ACE2 recombinant proteins disclosed herein may comprise an ACE polypeptide that is at least about 80% (e.g., about 85%, about 90%, about 95%, or about 98%) sequence identity, individually or collectively, as compared with SEQ ID NO: 1 or SEQ ID NO: 2. As used herein, “individually” means that one ACE polypeptide of a recombinant fusion protein disclosed herein shares the indicated sequence identity relative to the corresponding ACE polypeptide of the recombinant fusion protein. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mot Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, ACE2 recombinant proteins disclosed herein may comprise ACE polypeptide that is at least about 80% (e.g., about 85%, about 90%, about 95%, or about 98%) sequence identity, individually or collectively, as compared with SEQ ID NOs: 3-9. In some embodiments, ACE2 recombinant proteins disclosed herein may comprise ACE polypeptide that is at least about 90% sequence identity, individually or collectively, as compared with SEQ ID NOs: 3-9. In some embodiments, ACE2 recombinant proteins disclosed herein may comprise ACE polypeptide having any one of SEQ ID NOs: 3-9.

In some embodiments, ACE2 recombinant proteins disclosed herein may comprise ACE polypeptide that is at least about 80% (e.g., about 85%, about 90%, about 95%, or about 98%) sequence identity, individually or collectively, as compared with SEQ ID NO: 10. In some embodiments, ACE2 recombinant proteins disclosed herein may comprise ACE polypeptide that is at least about 90% sequence identity, individually or collectively, as compared with SEQ ID NO: 10. In some preferred embodiments, an ACE2 recombinant protein disclosed herein may comprise ACE polypeptide having SEQ ID NO: 10.

### (b) Methods of Preparing ACE2 Recombinant Proteins

Any of the ACE2 recombinant proteins disclosed herein can be made by any method known in the art. See, for example, Harlow and Lane, (1998) A Laboratory Manual, Cold Spring Harbor Laboratory, New York.

If desired, an ACE2 recombinant protein of interest may be sequenced and the polynucleotide sequence may then be cloned into a vector for expression or propagation. The sequence encoding the ACE2 recombinant protein of interest may be maintained in vector in a host cell and the host cell can then be expanded and frozen for future use. In some embodiments, the polynucleotide sequence can used for genetic manipulation to, e.g., improve the affinity or other characteristics of the ACE2 recombinant protein. In some aspects, the polynucleotide sequence can genetically manipulated to obtain greater affinity and/or specificity to the target protein and greater efficacy in binding to a spike protein of a coronavirus, thereby blocking entry of the virus into host cells via the ACE2 receptor. It will be apparent to one of skill in the art that one or more polynucleotide changes can be made to an ACE recombinant protein and still maintain its binding specificity to the target protein. In some aspects, the polynucleotide sequence can genetically manipulated to obtain greater stability of the ACE2 recombinant protein compared to an ACE2 recombinant protein not subjected to genetic manipulation. In some embodiments, a genetically manipulated polynucleotide sequence disclosed herein may confer increased stability of an ACE2 recombinant protein. In some embodiments, a genetically manipulated polynucleotide sequence disclosed herein may confer increased stability of an ACE2 recombinant protein in a solute, solvent, aqueous solution, and the like. In some embodiments, a genetically manipulated polynucleotide sequence disclosed herein may confer increased stability of an ACE2 recombinant protein during transport. In some embodiments, a genetically manipulated polynucleotide sequence disclosed herein may confer a decrease in aggregation of an ACE2 recombinant protein compared to an ACE2 recombinant protein not subjected to genetic manipulation. In some embodiments, a genetically manipulated polynucleotide sequence disclosed herein may confer stability of an ACE2 recombinant protein during nebulization. In some embodiments, a genetically manipulated polynucleotide sequence disclosed herein may confer stability of an ACE2 recombinant protein during aerosolization. In some embodiments, a genetically manipulated polynucleotide sequence disclosed herein may prevent protein aggregation of an ACE2 recombinant protein during nebulization. In some embodiments, a genetically manipulated polynucleotide sequence disclosed herein may prevent protein aggregation of an ACE2 recombinant protein during aerosolization.

In some embodiments, a polynucleotide sequence encoding for an ACE2 recombinant protein as disclosed herein may have a polynucleotide sequence having a sequence of any of the polynucleotides in Table 2.

TABLE 2

| Construct | DNA Sequences | SEQ ID |
|---|---|---|
| Name | (coding regions) | NO: |
| WT ACE2 | ATGAAATTTCTGGTCAATGTAGCCCTGGTATTCATGGTCGTCTACATC | 11 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |
| ACE2 T371X | ATGAAATTTCTGGTCAATGTAGCCCTGGTATTCATGGTCGTCTACATC | 12 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGXXXGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTICCAGGGTACCGGT | |
| | CATCACCACCACCACCATCACCATCATCATTGA | |
| ACE2 N149X | ATGAAATTTCTGGTCAATGTAGCCCTGGTATTCATGGTCGTCTACATC | 13 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCXXXGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |
| ACE2 F274X | ATGAAATTTCTGGTCAATGTAGCCCTGGTATTCATGGTCGTCTACATC | 14 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCXXXTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |
| ACE2 H345X | ATGAAATTTCTGGTCAATGTAGCCCTGGTATTCATGGTCGTCTACATC | 15 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCXXXCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |
| ACE2 F504X | ATGAAATTTCTGGTCAATGTAGCCCTGGTATTCATGGTCGTCTACATC | 16 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGXXXCATGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |
| ACE2 H505X | ATGAAATTTCTGGTCAATGTAGCCCTGGTATTCATGGTCGTCTACATC | 17 |
| | TCGTATATATATGCCTCGACAATAGAAGAACAAGCTAAGACTTTTCTC | |
| | GACAAATTTAACCATGAAGCAGAGGACCTCTTCTACCAGTCATCGTTG | |
| | GCATCGTGGAACTACAACACGAATATAACCGAGGAAAACGTTCAGAAT | |
| | ATGAACAACGCCGGCGATAAGTGGAGCGCCTTCCTTAAGGAGCAATCT | |
| | ACGCTGGCCCAGATGTATCCCTTGCAGGAGATTCAAAATCTTACGGTT | |
| | AAGCTTCAGCTGCAGGCCCTGCAGCAAAATGGTTCAAGCGTACTCAGT | |
| | GAAGATAAATCTAAGAGACTGAATACAATTCTCAACACCATGAGCACC | |
| | ATTTACTCTACAGGAAAGGTGTGCAACCCCGATAATCCACAAGAGTGC | |
| | CTGTTGCTGGAGCCTGGCCTCAATGAAATTATGGCAAACAGTCTTGAC | |
| | TACAATGAGAGGCTCTGGGCGTGGGAGTCTTGGAGGAGTGAGGTGGGA | |
| | AAGCAGCTGCGCCCACTGTATGAAGAGTATGTAGTCCTTAAGAACGAG | |
| | ATGGCGCGCGCAAATCATTATGAGGATTATGGAGATTACTGGCGTGGC | |
| | GACTATGAAGTGAATGGCGTCGACGGATACGATTATTCGCGCGGCCAA | |
| | CTCATCGAGGATGTAGAGCACACTTTCGAAGAGATAAAGCCTTTGTAC | |
| | GAGCACCTTCACGCGTACGTTAGGGCCAAGCTTATGAATGCTTACCCA | |
| | TCTTATATTAGCCCTATCGGCTGCCTGCCTGCGCATCTGTTGGGAGAT | |
| | ATGTGGGGTCGCTTCTGGACCAACCTCTATTCTCTTACTGTCCCATTC | |
| | GGCCAAAAGCCAAATATCGACGTGACAGATGCCATGGTCGATCAAGCA | |
| | TGGGACGCCCAAAGGATATTCAAGGAAGCCGAAAAATTTTTTGTTAGT | |
| | GTCGGCCTCCCCAACATGACGCAAGGTTTCTGGGAGAATTCTATGCTC | |
| | ACGGATCCAGGTAACGTGCAGAAGGCCGTATGCCACCCCACAGCCTGG | |
| | GACTTGGGTAAAGGAGACTTCAGGATTTTGATGTGTACCAAAGTTACC | |
| | ATGGATGATTTTCTGACTGCACACCACGAAATGGGCCACATTCAGTAT | |
| | GATATGGCTTACGCTGCTCAACCATTTCTTCTTCGCAATGGAGCCAAC | |
| | GAGGGATTTCATGAAGCAGTAGGTGAAATAATGTCGCTGAGTGCGGCA | |
| | ACACCGAAGCACCTTAAGTCCATTGGACTGCTCAGCCCCGACTTTCAG | |
| | GAAGATAACGAGACGGAGATAAACTTCCTGCTCAAGCAGGCACTCACG | |
| | ATCGTGGGAACGTTGCCTTTTACGTATATGCTTGAAAAGTGGAGATGG | |
| | ATGGTGTTCAAAGGCGAAATACCTAAAGACCAGTGGATGAAAAAGTGG | |
| | TGGGAAATGAAGAGAGAAATAGTCGGAGTCGTCGAACCCGTGCCGCAT | |
| | GATGAAACCTACTGCGATCCTGCCAGCTTGTTTXXXGTGAGTAATGAT | |
| | TATAGTTTCATACGCTACTATACTCGCACGTTGTACCAGTTTCAATTT | |
| | CAGGAGGCCCTGTGTCAAGCGGCAAAGCATGAGGGACCGCTGCACAAG | |
| | TGCGACATAAGTAATAGCACTGAGGCTGGACAAAAGCTGTTTAACATG | |
| | CTGCGTCTCGGAAAGAGTGAGCCTTGGACTCTCGCACTGGAAAATGTC | |
| | GTTGGCGCGAAAAACATGAATGTACGTCCGCTTCTGAATTACTTTGAG | |
| | CCATTGTTTACATGGTTGAAAGATCAGAATAAAAATAGTTTTGTGGGC | |
| | TGGTCAACGGATTGGAGCCCTTACGCTGATCAATCTATAAAGGTGAGA | |
| | ATATCCCTTAAGAGTGCGTTGGGAGATAAGGCATACGAGTGGAACGAC | |
| | AATGAAATGTACTTGTTCCGCTCTTCAGTAGCATACGCTATGAGGCAG | |
| | TATTTCCTTAAAGTAAAAAATCAGATGATCTTGTTTGGCGAAGAAGAT | |
| | GTCCGCGTTGCTAACCTTAAGCCAAGAATTTCGTTCAACTTTTTTGTT | |
| | ACCGCACCAAAGAATGTCTCAGACATCATACCCCGCACTGAAGTAGAA | |
| | AAAGCGATTAGAATGTCGCGTAGTCGTATCAACGACGCGTTTAGGCTC | |
| | AACGACAATTCGCTGGAATTCCTGGGAATTCAGCCCACATTGGGCCCT | |
| | CCAAACCAGCCTCCGGTGTCGGAGAATCTCTACTTCCAGGGTACCGGT | |
| | CATCACCACCACCACCATCACCATCATCATTGA | |

In some embodiments, a polynucleotide encoding an ACE2 recombinant protein disclosed herein may have a polynucleotide sequence having a sequence of any of the polynucleotides in Table 2 wherein “XXX” denotes the site of a possible mutation for the polynucleotide sequence. In some embodiments, possible mutation(s) to any of the polynucleotide sequences in Table 2 at the “XXX” site can be: TTT; TTC; TTA; TTG; OTT; CTC; CTA; CTG; ATT; ATC; ATA; ATG; GTT; GTC; GTA; GTG; TCT; TCC; TCA; TCG; AGT; AGC; CCT; CCC; CCA; CCG; ACT; ACC; ACA; ACG; GCT; GCC; GCA; GCT; TAT; TAC; CAT; CAC; CAA; CAG; AAT; AAC; AAA; AAG; GAT; GAC; GAA; GAG; TGT; TGC; TGG; CGT; CGC; CGA; CGG; AGA; AGG; GGT; GGC; GGA; or GGG.

In some preferred embodiments, a polynucleotide encoding an ACE2 recombinant protein disclosed herein may have a polynucleotide sequence as follows:

ACE2 T371W:
(SEQ ID NO: 18)
ATGAAATTTCTGGTCAATGTAGCCCTGGTATTCATGGTCGTCTACATCTCGTATATATATGCC
TCGACAATAGAAGAACAAGCTAAGACTTTTCTCGACAAATTTAACCATGAAGCAGAGGACCTC
TTCTACCAGTCATCGTTGGCATCGTGGAACTACAACACGAATATAACCGAGGAAAACGTTCAG
AATATGAACAACGCCGGCGATAAGTGGAGCGCCTTCCTTAAGGAGCAATCTACGCTGGCCCAG
ATGTATCCCTTGCAGGAGATTCAAAATCTTACGGTTAAGCTTCAGCTGCAGGCCCTGCAGCAA
AATGGTTCAAGCGTACTCAGTGAAGATAAATCTAAGAGACTGAATACAATTCTCAACACCATG
AGCACCATTTACTCTACAGGAAAGGTGTGCAACCCCGATAATCCACAAGAGTGCCTGTTGCTG
GAGCCTGGCCTCAATGAAATTATGGCAAACAGTCTTGACTACAATGAGAGGCTCTGGGCGTGG
GAGTCTTGGAGGAGTGAGGTGGGAAAGCAGCTGCGCCCACTGTATGAAGAGTATGTAGTCCTT
AAGAACGAGATGGCGCGCGCAAATCATTATGAGGATTATGGAGATTACTGGCGTGGCGACTAT
GAAGTGAATGGCGTCGACGGATACGATTATTCGCGCGGCCAACTCATCGAGGATGTAGAGCAC
ACTTTCGAAGAGATAAAGCCTTTGTACGAGCACCTTCACGCGTACGTTAGGGCCAAGCTTATG
AATGCTTACCCATCTTATATTAGCCCTATCGGCTGCCTGCCTGCGCATCTGTTGGGAGATATG
TGGGGTCGCTTCTGGACCAACCTCTATTCTCTTACTGTCCCATTCGGCCAAAAGCCAAATATC
GACGTGACAGATGCCATGGTCGATCAAGCATGGGACGCCCAAAGGATATTCAAGGAAGCCGAA
AAATTTTTTGTTAGTGTCGGCCTCCCCAACATGACGCAAGGTTTCTGGGAGAATTCTATGCTC
ACGGATCCAGGTAACGTGCAGAAGGCCGTATGCCACCCCACAGCCTGGGACTTGGGTAAAGGA
GACTTCAGGATTTTGATGTGTACCAAAGTTACCATGGATGATTTTCTGtggGCACACCACGAA
ATGGGCCACATTCAGTATGATATGGCTTACGCTGCTCAACCATTTCTTCTTCGCAATGGAGCC
AACGAGGGATTTCATGAAGCAGTAGGTGAAATAATGTCGCTGAGTGCGGCAACACCGAAGCAC
CTTAAGTCCATTGGACTGCTCAGCCCCGACTTTCAGGAAGATAACGAGACGGAGATAAACTTC
CTGCTCAAGCAGGCACTCACGATCGTGGGAACGTTGCCTTTTACGTATATGCTTGAAAAGTGG
AGATGGATGGTGTTCAAAGGCGAAATACCTAAAGACCAGTGGATGAAAAAGTGGTGGGAAATG
AAGAGAGAAATAGTCGGAGTCGTCGAACCCGTGCCGCATGATGAAACCTACTGCGATCCTGCC
AGCTTGTTTCATGTGAGTAATGATTATAGTTTCATACGCTACTATACTCGCACGTTGTACCAG
TTTCAATTTCAGGAGGCCCTGTGTCAAGCGGCAAAGCATGAGGGACCGCTGCACAAGTGCGAC
ATAAGTAATAGCACTGAGGCTGGACAAAAGCTGTTTAACATGCTGCGTCTCGGAAAGAGTGAG
CCTTGGACTCTCGCACTGGAAAATGTCGTTGGCGCGAAAAACATGAATGTACGTCCGCTTCTG
AATTACTTTGAGCCATTGTTTACATGGTTGAAAGATCAGAATAAAAATAGTTTTGTGGGCTGG
TCAACGGATTGGAGCCCTTACGCTGATCAATCTATAAAGGTGAGAATATCCCTTAAGAGTGCG
TTGGGAGATAAGGCATACGAGTGGAACGACAATGAAATGTACTTGTTCCGCTCTTCAGTAGCA
TACGCTATGAGGCAGTATTTCCTTAAAGTAAAAAATCAGATGATCTTGTTTGGCGAAGAAGAT
GTCCGCGTTGCTAACCTTAAGCCAAGAATTTCGTTCAACTTTTTTGTTACCGCACCAAAGAAT
GTCTCAGACATCATACCCCGCACTGAAGTAGAAAAAGCGATTAGAATGTCGCGTAGTCGTATC
AACGACGCGTTTAGGCTCAACGACAATTCGCTGGAATTCCTGGGAATTCAGCCCACATTGGGC
CCTCCAAACCAGCCTCCGGTGTCGGAGAATCTCTACTTCCAGGGTACCGGTCATCACCACCAC
CACCATCACCATCATCATTGA

In some embodiments, a polynucleotide sequence encoding an ACE2 recombinant protein disclosed herein may have a polynucleotide sequence that is at least about 80% (e.g., about 85%, about 90%, about 95%, or about 98%) sequence identity, individually or collectively, as compared with SEQ ID NO: 11. As used herein, “individually” means that one polynucleotide sequence herein shares the indicated sequence identity relative to the corresponding polynucleotide encoding an ACE polypeptide of the recombinant fusion protein. In some embodiments, ACE2 recombinant proteins disclosed herein may be encoded from a polynucleotide sequence that is at least about 80% (e.g., about 85%, about 90%, about 95%, or about 98%) sequence identity, individually or collectively, as compared with SEQ ID NOs: 11-17. In some embodiments, ACE2 recombinant proteins disclosed herein may be encoded from a polynucleotide sequence that is at least about 90% sequence identity, individually or collectively, as compared with SEQ ID NOs: 11-17. In some embodiments, ACE2 recombinant proteins disclosed herein may be encoded from a polynucleotide sequence having any one of SEQ ID NOs: 11-17.

In some embodiments, ACE2 recombinant proteins disclosed herein may be encoded from a polynucleotide sequence that is at least about 80% (e.g., about 85%, about 90%, about 95%, or about 98%) sequence identity, individually or collectively, as compared with SEQ ID NO: 18. In some embodiments, ACE2 recombinant proteins disclosed herein may be encoded from a polynucleotide sequence that is at least about 90% sequence identity, individually or collectively, as compared with SEQ ID NO: 18. In preferred embodiments, an exemplary ACE2 recombinant protein disclosed herein may be encoded from a polynucleotide sequence having SEQ ID NO: 18.

In certain embodiments, ACE2 recombinant proteins disclosed herein may be PEGylated. PEGylation (or pegylation) as used herein refers to the process of both covalent and non-covalent attachment or amalgamation of polyethylene glycol (PEG, in pharmacy called macrogol) polymer chains to molecules and macrostructures, such as a drug, therapeutic protein or vesicle, which is then described as PEGylated. The addition of polyethelene glycol (PEG) molecules can improve the pharmacokinetic and pharmacodynamic properties of any one of the ACE2 recombinant proteins disclosed herein. Polyethylene glycol, or “PEG” is a biologically inert, water soluble polymer that can be represented as linked to the polypeptide as formula:

XO—(CH₂CH₂O)ₙ—CH₂CH₂—Y
where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). The PEG can be linear or branched. Various forms of PEG including various functionalized derivatives are commercially available. Any of PEG or variant thereof can be contemplated for PEGylation of the ACE2 recombinant proteins disclosed herein without limitation. In some embodiments, any one of the ACE2 recombinant proteins having SEQ ID NOs: 1-10 as disclosed herein may be PEGylated.

In some embodiments, ACE2 recombinant proteins disclosed herein may be PEGylated in a non-specific manner. In some embodiments, ACE2 recombinant proteins disclosed herein may be PEGylated in a site-specific manner. Non-limiting methods used for site-specific conjugation of PEG suitable for use herein include N-terminal PEGylation, thiol and bridging PEGylation, chemical PEGylation, histidine tags, enzymatic PEGylation, and the like. In some embodiments, ACE2 recombinant proteins disclosed herein may be PEGylated in a site-specific manner by genetically encoding a single cysteine residue into the amino acid sequence. In accordance with these embodiments, ACE2 recombinant proteins disclosed herein may be PEGylated by reacting at least one free cysteine with a maleimide and/or bromide group attached to a PEG moiety. In accordance with these embodiments, cysteine-reactive PEG reagents that can be used to modify one or more introduced cysteines in a ACE2 recombinant protein disclosed herein may be methyl-PEG₁₂-maleimide, methyl-PEG₂₄-maleimide, methyl-PEG₃₆-maleimide, methyl-PEG₄₅-maleimide, methyl-PEG₁₂-bromide, or any combination thereof.

In certain embodiments, ACE2 recombinant proteins disclosed herein may comprise at least one cysteine mutation. In some embodiments, ACE2 recombinant proteins disclosed herein may comprise at least one cysteine mutation in a position of the protein predicted to be solvent-accessible. In some embodiments, ACE2 recombinant proteins disclosed herein may comprise at least one cysteine mutation near the ectodomain N-terminus, near the middle of the ectodomain, near the ectodomain C-terminus, or any combination thereof. In some embodiments, ACE2 recombinant proteins disclosed herein may comprise at least one cysteine mutation within about 50 amino acids from the ectodomain N-terminus. In some embodiments, ACE2 recombinant proteins disclosed herein may comprise at least one cysteine mutation within about 50 amino acids from the ectodomain C-terminus. In some embodiments, ACE2 recombinant proteins disclosed herein may comprise at least one cysteine mutation within about 50 amino acids from the ectodomain C-terminus of a polypeptide having a SEQ ID NO: 1-10 or encoded by a polynucleotide have a SEQ ID NO: 11-18.

In some embodiments, an ACE2 recombinant protein herein to be PEGylated by reacting at least one free cysteine with a maleimide and/or bromide group attached to a PEG moiety may have a polypeptide sequence and/or may be encoded from a polynucleotide sequence of Table 3.

TABLE 3

| | | SEQ ID |
|---|---|---|
| Construct Name | Amnio Acid/DNA Sequences | NO: |
| ACE2 T371W/G732C | MKFLVNVALVFMVVYISYIYASTIEEQAKTFLDKFNHEAEDLF | 22 |
| | YQSSLASWNYNTNITEENVQNMNNAGDKWSAFLKEQSTLAQMY | |
| | PLQEIQNLTVKLQLQALQQNGSSVLSEDKSKRLNTILNTMSTI | |
| | YSTGKVCNPDNPQECLLLEPGLNEIMANSLDYNERLWAWESWR | |
| | SEVGKQLRPLYEEYVVLKNEMARANHYEDYGDYWRGDYEVNGV | |
| | DGYDYSRGQLIEDVEHTFEEIKPLYEHLHAYVRAKLMNAYPSY | |
| | ISPIGCLPAHLLGDMWGRFWTNLYSLTVPFGQKPNIDVTDAMV | |
| | DQAWDAQRIFKEAEKFFVSVGLPNMTQGFWENSMLTDPGNVQK | |
| | AVCHPTAWDLGKGDFRILMCTKVTMDDFLWAHHEMGHIQYDMA | |
| | YAAQPFLLRNGANEGFHEAVGEIMSLSAATPKHLKSIGLLSPD | |
| | FQEDNETEINFLLKQALTIVGTLPFTYMLEKWRWMVFKGEIPK | |
| | DQWMKKWWEMKREIVGVVEPVPHDETYCDPASLFHVSNDYSFI | |
| | RYYTRTLYQFQFQEALCQAAKHEGPLHKCDISNSTEAGQKLEN | |
| | MLRLGKSEPWTLALENVVGAKNMNVRPLLNYFEPLFTWLKDQN | |
| | KNSFVGWSTDWSPYADQSIKVRISLKSALGDKAYEWNDNEMYL | |
| | FRSSVAYAMRQYFLKVKNQMILFGEEDVRVANLKPRISENFFV | |
| | TAPKNVSDIIPRTEVEKAIRMSRSRINDAFRLNDNSLEFLGIQ | |
| | PTLCPPNQPPVSENLYFQGTGHHHHHHHHHH* | |
| ACE2 T371W/S740C | MKFLVNVALVFMVVYISYIYASTIEEQAKTFLDKENHEAEDLF | 23 |
| | YQSSLASWNYNTNITEENVQNMNNAGDKWSAFLKEQSTLAQMY | |
| | PLQEIQNLTVKLQLQALQQNGSSVLSEDKSKRLNTILNTMSTI | |
| | YSTGKVCNPDNPQECLLLEPGLNEIMANSLDYNERLWAWESWR | |
| | SEVGKQLRPLYEEYVVLKNEMARANHYEDYGDYWRGDYEVNGV | |
| | DGYDYSRGQLIEDVEHTFEEIKPLYEHLHAYVRAKLMNAYPSY | |
| | ISPIGCLPAHLLGDMWGRFWTNLYSLTVPFGQKPNIDVTDAMV | |
| | DQAWDAQRIFKEAEKFFVSVGLPNMTQGFWENSMLTDPGNVQK | |
| | AVCHPTAWDLGKGDFRILMCTKVTMDDFLWAHHEMGHIQYDMA | |
| | YAAQPFLLRNGANEGFHEAVGEIMSLSAATPKHLKSIGLLSPD | |
| | FQEDNETEINFLLKQALTIVGTLPFTYMLEKWRWMVFKGEIPK | |
| | DQWMKKWWEMKREIVGVVEPVPHDETYCDPASLFHVSNDYSFI | |
| | RYYTRTLYQFQFQEALCQAAKHEGPLHKCDISNSTEAGQKLEN | |
| | MLRLGKSEPWTLALENVVGAKNMNVRPLLNYFEPLFTWLKDQN | |
| | KNSFVGWSTDWSPYADQSIKVRISLKSALGDKAYEWNDNEMYL | |
| | FRSSVAYAMRQYFLKVKNQMILFGEEDVRVANLKPRISENFFV | |
| | TAPKNVSDIIPRTEVEKAIRMSRSRINDAFRLNDNSLEFLGIQ | |
| | PTLGPPNQPPVCENLYFQGTGHHHHHHHHHH* | |
| ACE2 T371W/G732C | ATGAAATTTCTGGTCAATGTAGCCCTGGTATTCATGGTCGTCT | 24 |
| | ACATCTCGTATATATATGCCTCGACAATAGAAGAACAAGCTAA | |
| | GACTTTTCTCGACAAATTTAACCATGAAGCAGAGGACCTCTTC | |
| | TACCAGTCATCGTTGGCATCGTGGAACTACAACACGAATATAA | |
| | CCGAGGAAAACGTTCAGAATATGAACAACGCCGGCGATAAGTG | |
| | GAGCGCCTTCCTTAAGGAGCAATCTACGCTGGCCCAGATGTAT | |
| | CCCTTGCAGGAGATTCAAAATCTTACGGTTAAGCTTCAGCTGC | |
| | AGGCCCTGCAGCAAAATGGTTCAAGCGTACTCAGTGAAGATAA | |
| | ATCTAAGAGACTGAATACAATTCTCAACACCATGAGCACCATT | |
| | TACTCTACAGGAAAGGTGTGCAACCCCGATAATCCACAAGAGT | |
| | GCCTGTTGCTGGAGCCTGGCCTCAATGAAATTATGGCAAACAG | |
| | TCTTGACTACAATGAGAGGCTCTGGGCGTGGGAGTCTTGGAGG | |
| | AGTGAGGTGGGAAAGCAGCTGCGCCCACTGTATGAAGAGTATG | |
| | TAGTCCTTAAGAACGAGATGGCGCGCGCAAATCATTATGAGGA | |
| | TTATGGAGATTACTGGCGTGGCGACTATGAAGTGAATGGCGTC | |
| | GACGGATACGATTATTCGCGCGGCCAACTCATCGAGGATGTAG | |
| | AGCACACTTTCGAAGAGATAAAGCCTTTGTACGAGCACCTTCA | |
| | CGCGTACGTTAGGGCCAAGCTTATGAATGCTTACCCATCTTAT | |
| | ATTAGCCCTATCGGCTGCCTGCCTGCGCATCTGTTGGGAGATA | |
| | TGTGGGGTCGCTTCTGGACCAACCTCTATTCTCTTACTGTCCC | |
| | ATTCGGCCAAAAGCCAAATATCGACGTGACAGATGCCATGGTC | |
| | GATCAAGCATGGGACGCCCAAAGGATATTCAAGGAAGCCGAAA | |
| | AATTTTTTGTTAGTGTCGGCCTCCCCAACATGACGCAAGGTTT | |
| | CTGGGAGAATTCTATGCTCACGGATCCAGGTAACGTGCAGAAG | |
| | GCCGTATGCCACCCCACAGCCTGGGACTTGGGTAAAGGAGACT | |
| | TCAGGATTTTGATGTGTACCAAAGTTACCATGGATGATTTTCT | |
| | GTGGGCACACCACGAAATGGGCCACATTCAGTATGATATGGCT | |
| | TACGCTGCTCAACCATTTCTTCTTCGCAATGGAGCCAACGAGG | |
| | GATTTCATGAAGCAGTAGGTGAAATAATGTCGCTGAGTGCGGC | |
| | AACACCGAAGCACCTTAAGTCCATTGGACTGCTCAGCCCCGAC | |
| | TTTCAGGAAGATAACGAGACGGAGATAAACTTCCTGCTCAAGC | |
| | AGGCACTCACGATCGTGGGAACGTTGCCTTTTACGTATATGCT | |
| | TGAAAAGTGGAGATGGATGGTGTTCAAAGGCGAAATACCTAAA | |
| | GACCAGTGGATGAAAAAGTGGTGGGAAATGAAGAGAGAAATAG | |
| | TCGGAGTCGTCGAACCCGTGCCGCATGATGAAACCTACTGCGA | |
| | TCCTGCCAGCTTGTTTCATGTGAGTAATGATTATAGTTTCATA | |
| | CGCTACTATACTCGCACGTTGTACCAGTTTCAATTTCAGGAGG | |
| | CCCTGTGTCAAGCGGCAAAGCATGAGGGACCGCTGCACAAGTG | |
| | CGACATAAGTAATAGCACTGAGGCTGGACAAAAGCTGTTTAAC | |
| | ATGCTGCGTCTCGGAAAGAGTGAGCCTTGGACTCTCGCACTGG | |
| | AAAATGTCGTTGGCGCGAAAAACATGAATGTACGTCCGCTTCT | |
| | GAATTACTTTGAGCCATTGTTTACATGGTTGAAAGATCAGAAT | |
| | AAAAATAGTTTTGTGGGCTGGTCAACGGATTGGAGCCCTTACG | |
| | CTGATCAATCTATAAAGGTGAGAATATCCCTTAAGAGTGCGTT | |
| | GGGAGATAAGGCATACGAGTGGAACGACAATGAAATGTACTTG | |
| | TTCCGCTCTTCAGTAGCATACGCTATGAGGCAGTATTTCCTTA | |
| | AAGTAAAAAATCAGATGATCTTGTTTGGCGAAGAAGATGTCCG | |
| | CGTTGCTAACCTTAAGCCAAGAATTTCGTTCAACTTTTTTGTT | |
| | ACCGCACCAAAGAATGTCTCAGACATCATACCCCGCACTGAAG | |
| | TAGAAAAAGCGATTAGAATGTCGCGTAGTCGTATCAACGACGC | |
| | GTTTAGGCTCAACGACAATTCGCTGGAATTCCTGGGAATTCAG | |
| | CCCACATTGTGCCCTCCAAACCAGCCTCCGGTGTCGGAGAATC | |
| | TCTACTTCCAGGGTACCGGTCATCACCACCACCACCATCACCA | |
| | TCATCATTGA | |
| ACE2 T371W/S740C | ATGAAATTTCTGGTCAATGTAGCCCTGGTATTCATGGTCGTCT | 25 |
| | ACATCTCGTATATATATGCCTCGACAATAGAAGAACAAGCTAA | |
| | GACTTTTCTCGACAAATTTAACCATGAAGCAGAGGACCTCTTC | |
| | TACCAGTCATCGTTGGCATCGTGGAACTACAACACGAATATAA | |
| | CCGAGGAAAACGTTCAGAATATGAACAACGCCGGCGATAAGTG | |
| | GAGCGCCTTCCTTAAGGAGCAATCTACGCTGGCCCAGATGTAT | |
| | CCCTTGCAGGAGATTCAAAATCTTACGGTTAAGCTTCAGCTGC | |
| | AGGCCCTGCAGCAAAATGGTTCAAGCGTACTCAGTGAAGATAA | |
| | ATCTAAGAGACTGAATACAATTCTCAACACCATGAGCACCATT | |
| | TACTCTACAGGAAAGGTGTGCAACCCCGATAATCCACAAGAGT | |
| | GCCTGTTGCTGGAGCCTGGCCTCAATGAAATTATGGCAAACAG | |
| | TCTTGACTACAATGAGAGGCTCTGGGCGTGGGAGTCTTGGAGG | |
| | AGTGAGGTGGGAAAGCAGCTGCGCCCACTGTATGAAGAGTATG | |
| | TAGTCCTTAAGAACGAGATGGCGCGCGCAAATCATTATGAGGA | |
| | TTATGGAGATTACTGGCGTGGCGACTATGAAGTGAATGGCGTC | |
| | GACGGATACGATTATTCGCGCGGCCAACTCATCGAGGATGTAG | |
| | AGCACACTTTCGAAGAGATAAAGCCTTTGTACGAGCACCTTCA | |
| | CGCGTACGTTAGGGCCAAGCTTATGAATGCTTACCCATCTTAT | |
| | ATTAGCCCTATCGGCTGCCTGCCTGCGCATCTGTTGGGAGATA | |
| | TGTGGGGTCGCTTCTGGACCAACCTCTATTCTCTTACTGTCCC | |
| | ATTCGGCCAAAAGCCAAATATCGACGTGACAGATGCCATGGTC | |
| | GATCAAGCATGGGACGCCCAAAGGATATTCAAGGAAGCCGAAA | |
| | AATTTTTTGTTAGTGTCGGCCTCCCCAACATGACGCAAGGTTT | |
| | CTGGGAGAATTCTATGCTCACGGATCCAGGTAACGTGCAGAAG | |
| | GCCGTATGCCACCCCACAGCCTGGGACTTGGGTAAAGGAGACT | |
| | TCAGGATTTTGATGTGTACCAAAGTTACCATGGATGATTTTCT | |
| | GTGGGCACACCACGAAATGGGCCACATTCAGTATGATATGGCT | |
| | TACGCTGCTCAACCATTTCTTCTTCGCAATGGAGCCAACGAGG | |
| | GATTTCATGAAGCAGTAGGTGAAATAATGTCGCTGAGTGCGGC | |
| | AACACCGAAGCACCTTAAGTCCATTGGACTGCTCAGCCCCGAC | |
| | TTTCAGGAAGATAACGAGACGGAGATAAACTTCCTGCTCAAGC | |
| | AGGCACTCACGATCGTGGGAACGTTGCCTTTTACGTATATGCT | |
| | TGAAAAGTGGAGATGGATGGTGTTCAAAGGCGAAATACCTAAA | |
| | GACCAGTGGATGAAAAAGTGGTGGGAAATGAAGAGAGAAATAG | |
| | TCGGAGTCGTCGAACCCGTGCCGCATGATGAAACCTACTGCGA | |
| | TCCTGCCAGCTTGTTTCATGTGAGTAATGATTATAGTTTCATA | |
| | CGCTACTATACTCGCACGTTGTACCAGTTTCAATTTCAGGAGG | |
| | CCCTGTGTCAAGCGGCAAAGCATGAGGGACCGCTGCACAAGTG | |
| | CGACATAAGTAATAGCACTGAGGCTGGACAAAAGCTGTTTAAC | |
| | ATGCTGCGTCTCGGAAAGAGTGAGCCTTGGACTCTCGCACTGG | |
| | AAAATGTCGTTGGCGCGAAAAACATGAATGTACGTCCGCTTCT | |
| | GAATTACTTTGAGCCATTGTTTACATGGTTGAAAGATCAGAAT | |
| | AAAAATAGTTTTGTGGGCTGGTCAACGGATTGGAGCCCTTACG | |
| | CTGATCAATCTATAAAGGTGAGAATATCCCTTAAGAGTGCGTT | |
| | GGGAGATAAGGCATACGAGTGGAACGACAATGAAATGTACTTG | |
| | TTCCGCTCTTCAGTAGCATACGCTATGAGGCAGTATTTCCTTA | |
| | AAGTAAAAAATCAGATGATCTTGTTTGGCGAAGAAGATGTCCG | |
| | CGTTGCTAACCTTAAGCCAAGAATTTCGTTCAACTTTTTTGTT | |
| | ACCGCACCAAAGAATGTCTCAGACATCATACCCCGCACTGAAG | |
| | TAGAAAAAGCGATTAGAATGTCGCGTAGTCGTATCAACGACGC | |
| | GTTTAGGCTCAACGACAATTCGCTGGAATTCCTGGGAATTCAG | |
| | CCCACATTGGGCCCTCCAAACCAGCCTCCGGTGTGCGAGAATC | |
| | TCTACTTCCAGGGTACCGGTCATCACCACCACCACCATCACCA | |
| | TCATCATTGA | |

In Table 3, Bold indicates secretion peptide sequence, Bold underline indicates residues that differ from WT ACE2 ectodomain, Bold italics indicates purification epitope tag introduced at the C-terminus, and * indicates stop codon.

In some embodiments, an ACE2 recombinant protein herein to be PEGylated by reacting at least one free cysteine with a maleimide and/or bromide group attached to a PEG moiety may have a polypeptide sequence that is at least about 90% sequence identity, individually or collectively, as compared with SEQ ID NOs: 22-23. In some embodiments, an ACE2 recombinant protein herein to be PEGylated by reacting at least one free cysteine with a maleimide and/or bromide group attached to a PEG moiety may be encoded from a polynucleotide sequence that is at least about 90% sequence identity, individually or collectively, as compared with SEQ ID NOs: 24-25. In some embodiments, an ACE2 recombinant protein herein to be PEGylated by reacting at least one free cysteine with a maleimide and/or bromide group attached to a PEG moiety may have a polypeptide sequence corresponding to SEQ ID NOs: 22-23. In some embodiments, an ACE2 recombinant protein herein to be PEGylated by reacting at least one free cysteine with a maleimide and/or bromide group attached to a PEG moiety may be encoded from a polynucleotide sequence corresponding to SEQ ID NOs: 24-25.

In certain embodiments, ACE2 recombinant proteins herein modified by cysteine-PEGylation may retain at least about 25%, at least about 50%, between about 50% to about 75%, or about 100% of the biological activity associated with the unmodified protein. In some embodiments, cysteine-PEGylated ACE2 recombinant proteins herein may have a higher half-life (t_{1/2}) relative to the half-life of the unmodified protein from which it was derived following intranasal administration to a subject in need thereof. In accordance with these embodiments, the half-life of the cysteine-PEGylated protein may be enhanced by at least about 1.5-fold to about 2-fold, about 2-fold, about 3-fold, about 4-fold, about 5-fold, about 6-fold, about 7-fold, about 8-fold, about 9-fold, about 10-fold, about 20-fold, about 40-fold, about 60-fold, about 80-fold, or about 100-fold relative to the half-life of the unmodified parent protein.

Genetically engineered ACE2 recombinant proteins disclosed herein, such as humanized ACE2 recombinant proteins, chimeric ACE2 recombinant proteins, homodimer ACE2 recombinant proteins, dimerized ACE2 recombinant proteins, and the like can be produced via, e.g., conventional recombinant technology. In some embodiments, DNA encoding an ACE2 recombinant protein specific to a target protein can be readily isolated and sequenced using conventional procedures. Once isolated, the DNA may be placed into one or more expression vectors, which are then transfected into host cells such as E. coli cells, simian COS cells, Chinese hamster ovary (CHO) cells, Human Embryotic Kindey (HEK) 293 cells or myeloma cells that do not otherwise produce the ACE2 recombinant proteins disclosed herein.

In some embodiments, ACE2 recombinant proteins disclosed herein can be prepared by recombinant technology as exemplified below. Nucleic acids encoding the ACE2 recombinant protein as described herein can be cloned into one expression vector, each nucleotide sequence being in operable linkage to a suitable promoter. In some embodiments, each of the nucleotide sequences encoding ACE2 recombinant proteins disclosed herein can be in operable linkage to a distinct prompter. Generally, a nucleic acid sequence encoding an ACE2 recombinant disclosed herein can be cloned into a suitable expression vector in operable linkage with a suitable promoter using methods known in the art. In some embodiments, nucleotide sequences and/or vectors can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers can contain nucleic acid sequences that correspond to a particular restriction site in the vector. The selection of expression vectors/promoter can depend on the type of host cells for use in producing an ACE recombinant protein herein.

A variety of promoters can be used for expression of ACE recombinant proteins described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters can include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems can include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sol. USA, 89:5547-5551 (1992); Shockett et al., Proc. Natl. Acad. Sci. USA, 92:6522-6526 (1995)), to achieve its regulatable effects.

Additionally, vectors disclosed herein can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful herein can include, but are not limited to, human collagen I polyadenylation signal, human collagen II polyadenylation signal, and SV40 polyadenylation signal.

One or more vectors (e.g., expression vectors) comprising nucleic acids encoding any of the ACE2 recombinant proteins disclosed herein may be introduced into suitable host cells for producing the ACE2 recombinant proteins. The host cells can be cultured under suitable conditions for expression of an ACE recombinant protein or any polypeptide chain thereof. Such ACE2 recombinant proteins or polypeptide chains thereof can be recovered by the cultured cells (e.g., from the cells or the culture supernatant) via a conventional method, e.g., affinity purification. If necessary, ACE2 recombinant proteins disclosed herein can be incubated under suitable conditions for a suitable period of time allowing for production of the ACE2 recombinant protein.

In some embodiments, methods for preparing an ACE2 recombinant protein disclosed herein can involve a recombinant expression vector that encodes all components of the ACE2 recombinant proteins as also described herein. The recombinant expression vector can be introduced into a suitable host cell (e.g., a HEK293T cell or a dhfr− CHO cell) by a conventional method, e.g., calcium phosphate-mediated transfection. Positive transformant host cells can be selected and cultured under suitable conditions allowing for the expression of ACE recombinant proteins which can be recovered from the cells or from the culture medium. When necessary, the ACE2 recombinant proteins recovered from the host cells can be incubated under suitable conditions allowing for the formation of de ACE2 recombinant protein dimers.

Standard molecular biology techniques can be used to prepare the recombinant expression vector, transfect the host cells, select for transformants, culture the host cells and recovery of the ACE2 recombinant proteins from the culture medium. In some embodiments, some ACE2 recombinant proteins disclosed herein can be isolated by affinity chromatography with a Protein A or Protein G coupled matrix. In some embodiments, ACE2 recombinant proteins disclosed herein may include a tag and the like to isolate and/or purify the ACE2 recombinant protein. In some embodiments, ACE2 recombinant proteins disclosed herein may be subjected to enzymatic cleavage to remove a tag, linker, signaling peptide, or a combination thereof after purification.

Any of the nucleic acids encoding the ACE2 recombinant proteins as disclosed herein, vectors (e.g., expression vectors) containing such; and host cells comprising the vectors are within the scope of the present disclosure.

### III. Therapeutic Applications

Any of the ACE2 recombinant proteins disclosed herein can be used for therapeutic, diagnostic, and/or research purposes, all of which are within the scope of the present disclosure.

### (a) Pharmaceutical Compositions

The ACE2 recombinant proteins, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present disclosure can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, a pharmaceutical composition described herein can include liposomes containing the ACE2 recombinant proteins (or the encoding nucleic acids) disclosed herein which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The ACE2 recombinant proteins disclosed herein, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In some embodiments, a pharmaceutical composition described herein can be formulated in sustained-release format. Suitable examples of sustained-release preparations include semipermeable matrices of solid hydrophobic polymers containing the protein, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions disclosed herein to be used for in vivo administration should be sterile. This can be readily accomplished by, for example, filtration through sterile filtration membranes. Therapeutic ACE recombinant proteins compositions disclosed herein can be generally placed into a container having a sterile access port, for example, an intravenous solution bag or vial having a stopper pierceable by a hypodermic injection needle.

The pharmaceutical compositions disclosed herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient (e.g., the ACE2 recombinant proteins (or the encoding nucleic acids) disclosed herein) can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 mg to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween 20, Tween 40, Tween 60, Tween 80 or Tween 85) and other sorbitans (e.g., Span 20, Span 40, Span 60, Span 80 or Span 85). Compositions with a surface-active agent will conveniently comprise between about 0.05 and about 5% surface-active agent and can be between about 0.1% and about 2.5%. It will be appreciated that other ingredients may be added, for example mannitol or other pharmaceutically acceptable vehicles, if necessary.

Suitable emulsions may be prepared using commercially available fat emulsions, such as Intralipid, Liposyn, Infonutrol, Lipofundin and Lipiphysan. The active ingredient (e.g., the ACE2 recombinant proteins (or the encoding nucleic acids) disclosed herein) may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to about 20% oil, for example, between about 5% and about 20%. The fat emulsion can comprise fat droplets between about 0.1 μm and 1.0 μm, particularly about 0.1 μm and 0.5 μm, and have a pH in the range of about 5.5 to about 8.0. The emulsion compositions can be those prepared by mixing an ACE recombinant protein with Intralipid or the components thereof (soybean oil, egg phospholipids, glycerol and water).

Pharmaceutical compositions for inhalation or insufflation disclosed herein can include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions disclosed herein can be administered by the oral or nasal respiratory route for local or systemic effect. Solution, suspension or powder compositions may be administered, preferably orally or nasally, from devices which deliver the formulation in an appropriate manner. In some embodiments, pharmaceutical compositions disclosed herein can be formulated for delivery via an aerosol delivery device. In some embodiments, an aerosol delivery device suitable for use here can be a nebulizer, a pressurized metered-dose inhaler (pMDI), or a dry powder inhaler (DPI).

Pharmaceutical compositions disclosed herein for inhalation can be formulated for administration by a nebulizer. Nebulizers can provide a means of administering the pharmaceutical compositions disclosed herein to the airways of a subject in need thereof while the subject breathes at an approximately normal rate. Nebulizers are particularly suitable for subjects who are unable to inhale at the much higher rates required for administration of drugs via metered dose inhalers or dry powder inhalers and for subjects who cannot for whatever reason coordinate the activation of the metered dose inhaler with their inhalation of breath.

A nebulized solution as disclosed herein can be dispersed in air to form an aerosol. In some embodiments, a nebulizer as disclosed herein can generate liquid droplets suitable for inhalation into the respiratory tract of a subject. In some embodiments, a nebulizer as disclosed herein can generate liquid droplets suitable for inhalation into at least one lung of a subject. As used herein, the droplet size and/or the particle size of a generated aerosol can be evaluated by mass median diameter (MMD) of the droplet. For aerosol delivery to the respiratory tract (and/or a lung), an aerosol disclosed herein can include droplets and/or particles having a MMD below about 10.0 μm. In some embodiments, an aerosol formed from a nebulized solution disclosed herein can include droplets and/or particles having a MMD of less than about 10.0 μm to less than about 0.5 μm. In some examples, an aerosol formed from a nebulized solution disclosed herein can include droplets and/or particles having a MMD below about 10.0 μm, below about 5.0 μm, below about 2.0 μm, below about 1.0 μm, or below about 0.5 μm. In some embodiments, an aerosol disclosed herein can include droplets and/or particles having a MMD below about 0.5 μm, below about 1.0 μm, below about 1.5 μm, below about 2.0 μm, below about 2.5 μm, below about 3.0 μm, below about 3.5 μm, below about 4.0 μm, below about 4.5 μm, below about 5.5 μm, below about 6.0 μm, below about 6.5 μm, below about 7.0 μm, below about 7.5 μm, below about 8.0 μm, below about 8.5 μm, below about 9.0 μm, below about 9.5 μm, or below about 10.0 μm

In some embodiments, an aerosol formed from a nebulized solution disclosed herein can have droplets and/or particles of uniform size. In some embodiments, an aerosol formed from a nebulized solution disclosed herein can have droplets and/or particles of at least two different sizes. In some embodiments, an aerosol formed from a nebulized solution disclosed herein can have greater than about 5% to about 90% of droplets and/or particles having a MMD smaller than about 10 μm. In some embodiments, an aerosol formed from a nebulized solution disclosed herein can have greater than about 5% to about 90% of droplets and/or particles having a MMD smaller than about 5.0 μm. In some embodiments, an aerosol formed from a nebulized solution disclosed herein can have greater than about 5% to about 90% of droplets and/or particles having a MMD smaller than about 2.0 μm. In some embodiments, an aerosol formed from a nebulized solution disclosed herein can have greater than about 5% to about 90% of droplets and/or particles having a MMD smaller than about 1.0 μm. In some embodiments, an aerosol formed from a nebulized solution disclosed herein can have greater than about 5% to about 90% of droplets and/or particles having a MMD smaller than about 0.5 μm.

In some embodiments, pharmaceutical compositions herein can include sterile pharmaceutically acceptable solvents that may be nebulized. Nebulization can be achieved by any nebulizer known in the art. A variety of nebulizers can be used with the pharmaceutical compositions herein, including but not limited to ultrasonic nebulizers, jet nebulizers and breath-actuated nebulizers. Nebulized solutions herein may be breathed directly from the nebulizing device or the nebulizing device may be attached to a mouthpiece, face mask, tent or intermittent positive pressure breathing machine. In some embodiments, nebulizers of use herein can further have a baffle to remove larger droplets from the mist by impaction. In some embodiments, a reservoir volume of the nebulizer can range from about 1.0 mL to about 10.0 mL, about 2.0 mL to about 9.0 mL, or about 3.0 mL to about 8.0 mL. In some embodiments, a reservoir volume of the nebulizer may be about 1.0 mL, about 2.0 mL, about 3.0 mL, about 4.0 mL, about 5.0 mL, about 6.0 mL, about 7.0 mL, about 8.0 mL, about 9.0 mL, or about 10.0 mL.

Nebulizers of use herein can use, but are not limited to using, compressed air and or other gases (i.e., a “jet nebulizer”), ultrasonic waves, or a vibrating mesh to create a mist of the droplets. A jet nebulizer includes tubing connected to an air/gas compressor, which causes compressed air or oxygen to flow at a high velocity through a liquid composition to turn it into an aerosol, which can then be inhaled by the subject. An ultrasonic wave nebulizer encompasses an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Examples of ultrasonic wave nebulizers include, but are not limited to, the Omron NE-U17 and the Beurer Nebulizer IH30. A vibrating mesh nebulizer includes a mesh/membrane with about 1000 to about 7000 holes that vibrates at the top of a liquid reservoir and thereby pressures out a mist of very fine aerosol droplets through the holes in the mesh/membrane. Examples of vibrating mesh nebulizers include, but are not limited to, NEB400 Mini Mesh Nebulizer (Sunset Healthcare Solutions), eFlow (PARI Medical Ltd.), i-Neb (Respironics Respiratory Drug Delivery Ltd), Nebulizer IH50 (Beurer Ltd.), AeroNeb Go (Aerogen Ltd.), InnoSpire Go (Respironics Respiratory Drug Delivery Ltd), Mesh Nebulizer (Shenzhen Homed Medical Device Co, Ltd.), Portable Nebulizer (Microbase Technology Corporation) and Airworks (Convexity Scientific LLC). In some embodiments, the mesh or membrane of a vibrating mesh nebulizer used herein can be made to vibrate by a piezoelectric element. In some embodiments, the mesh or membrane of vibrating mesh nebulizers used herein can be made to vibrate by ultrasound.

### (b) Therapeutic Applications

To practice the methods disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for methods of administration disclosed herein. Liquid formulations can be directly nebulized and lyophilized powder can be nebulized after reconstitution. Alternatively, the ACE2 recombinant proteins (or the encoding nucleic acids) disclosed herein can be aerosolized using a fluorocarbon formulation and a metered dose inhaler, or inhaled as a lyophilized and milled powder. In preferred embodiments, ACE2 recombinant proteins disclosed herein can be aerosolized for suitable administration.

The subject to be treated by the methods described herein can be a mammal, more preferably a human. Mammals include, but are not limited to, farm animals, sport animals, pets, primates, horses, dogs, cats, mice and rats. In some embodiments, a subject may have, be at risk for, or be suspected of having, a target disease/disorder characterized by a viral infection wherein the virus employs ACE2 receptor binding for at least one viral activity. In some embodiments, a subject may have, be at risk for, or be suspected of having, a target disease/disorder characterized by a coronavirus infection. In accordance with these embodiments, the coronavirus may be SARS-CoV-2, severe acute respiratory syndrome coronavirus (SARS-CoV), or Middle East respiratory syndrome coronavirus (MERS-CoV). The coronavirus may also be human coronavirus 229E, NL63, OC43, or HKU1. In some embodiments, the coronavirus is SARS-CoV-2. The target disease/disorder may be SARS, MERS, or COVID-19. In some embodiments, the target disease/disorder is COVID-19.

A subject having a coronavirus infection or suspected of having the infection can be identified by routine medical examination, e.g., laboratory tests, organ functional tests, or CT scans. In some embodiments, a subject in need as disclosed herein can have a SARS-CoV-2 infection or is suspected of having such an infection. In some embodiments, a subject in need as disclosed herein can have COVID-19 or is suspected of having COVID-19.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the ACE2 recombinant protein (or the encoding nucleic acids) disclosed herein achieved the therapeutic effect would be evident to one of skill in the art. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, ACE2 recombinant proteins disclosed herein that are compatible with the human immune system, such as humanized fusion proteins or fully human proteins, may be used to prolong half-life of the ACE2 recombinant protein and to prevent the ACE2 recombinant protein being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an ACE2 recombinant protein may be appropriate. Various formulations and devices for achieving sustained release are known in the art.

In some embodiments, dosages for a ACE2 recombinant protein as described herein may be determined empirically in individuals who have been given one or more administration(s) of the an ACE recombinant protein. Individuals are given incremental dosages of the agonist. To assess efficacy of the agonist, an indicator of the disease/disorder can be followed.

Generally, for administration of any of the ACE2 recombinant proteins described herein, an initial candidate dosage can be about 1 mg/kg to about 30 mg/kg. For the purpose of the present disclosure, a typical daily dosage can range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen may comprise administering an initial dose of at least about 1 mg/kg of an ACE2 recombinant protein disclosed herein, followed by a weekly maintenance dose of at least about 1 mg/kg of an ACE2 recombinant protein disclosed herein, or followed by a maintenance dose of at least about 1 mg/kg of an ACE2 recombinant protein disclosed herein every other week. However, other dosage regimens may be useful, depending on the pattern of pharmacokinetic decay that the practitioner wishes to achieve. For example, dosing from one-four times a week is contemplated. In some embodiments, dosing ranging from about 1 mg/kg to about 30 mg/kg (such as about 1 mg/kg, about 5 mg/kg, about 10 mg/kg, about 15 mg/kg, about 20 mg/kg, about 25 mg/kg, about 30 mg/kg) of an ACE2 recombinant protein disclosed herein may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy can be monitored by conventional techniques and assays. The dosing regimen (including the ACE recombinant protein used) can vary over time.

In some embodiments, for an adult patient of normal weight, doses ranging from about 0.1 μg/kg to about 100 mg/kg of ACE2 recombinant protein as disclosed herein may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an ACE2 recombinant protein as described herein will depend on the specific peptides (or compositions thereof) employed, the type and severity of the disease/disorder, whether the ACE recombinant protein is administered for preventive or therapeutic purposes, previous therapy, the patient's clinical history and response to the agonist, and the discretion of the attending physician. Typically the clinician will administer an ACE2 recombinant protein, until a dosage is reached that achieves the desired result. In some embodiments, the desired result can be a decrease or complete inhibition of viral infection. In some embodiments, the desired result can be decrease or complete inhibition of coronavirus infection. In some embodiments, an ACE2 recombinant protein disclosed herein can decrease the rate of coronavirus infection by at least about 20% (e.g., about 30%, about 40%, about 45%, about 50%, about 60%, about 70%, about 80%, about 90%, about 95% or greater, including any increment therein) following administration to a subject in need thereof. In some embodiments, the desired result can be a decrease and/or complete inhibition of viral replication. In some embodiments, the desired result can be a decrease and/or complete inhibition of coronavirus viral replication. In some embodiments, an ACE2 recombinant protein disclosed herein can decrease the rate of coronavirus replication by at least about 20% (e.g., about 30%, about 40%, about 45%, about 50%, about 60%, about 70%, about 80%, about 90%, about 95% or greater, including any increment therein) following administration to a subject in need thereof. Methods of determining whether a dosage resulted in the desired result would be evident to one of skill in the art. Administration of one or more ACE2 recombinant proteins disclosed herein can be continuous or intermittent, depending, for example, upon the recipient's physiological condition, whether the purpose of the administration is therapeutic or prophylactic, and other factors known to skilled practitioners. The administration of an ACE2 recombinant protein disclosed herein may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, pharmaceutical compositions disclosed herein can be administered to the subject via injectable depot routes of administration such as using 1-, 3-, or 6-month depot injectable or biodegradable materials and methods. In some embodiments, pharmaceutical compositions disclosed herein may be administered intraocularly or intravitreally. In some embodiments, pharmaceutical compositions disclosed herein may be administered in an aerosolized form.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble ACE recombinant proteins disclosed herein can be administered by the drip method, whereby a pharmaceutical formulation containing an ACE recombinant protein disclosed herein and a physiologically acceptable excipient may be infused. Physiologically acceptable excipients may include, for example, 5% dextrose, 0.9% saline, Ringer's solution or other suitable excipients. Intramuscular preparations, e.g., a sterile formulation of a suitable soluble salt form of the ACE recombinant proteins herein, can be dissolved and administered in a pharmaceutical excipient such as Water-for-Injection, 0.9% saline, or 5% glucose solution.

In some embodiments, an ACE2 recombinant protein disclosed herein can be administered via site-specific or targeted local delivery techniques. Examples of site-specific or targeted local delivery techniques include various implantable depot sources of the protein or local delivery catheters, such as infusion catheters, an indwelling catheter, or a needle catheter, synthetic grafts, adventitial wraps, shunts and stents or other implantable devices, site specific carriers, direct injection, or direct application via inhalation.

In some embodiments, an ACE2 recombinant protein disclosed herein can be administered by direct application via inhalation. In some embodiments, an ACE2 recombinant protein disclosed herein can be administered via inhalation using a nebulizer. In some embodiments, an ACE2 recombinant protein disclosed herein can be administered using a nebulizer adapted for generating an aerosol at an effective flow rate. As used herein, an effective flow rate refers to the flow rate of the aerosol as it enters the respiratory system of the subject in need thereof. One of skill in the art will know to adjust the flow rate as needed to reach the subject's respiratory system. In some embodiments, an effective flow rate of an aerosol herein can be less than about 5 liters/minute to less than about 5 liters/minute.

In some embodiments, an ACE2 recombinant protein disclosed herein can be administered using a nebulizer, wherein the nebulizer can deliver at least about 10% to at least about 100% of the ACE2 recombinant protein to at least one of the subject's lungs. In some embodiments, an ACE2 recombinant protein disclosed herein can be administered using a nebulizer, wherein the nebulizer can deliver at least about 10%, at least about 25%, at least about 50%, at least about 75%, at least about 95%, or at least about 100% of the ACE2 recombinant protein to at least one of the subject's lungs.

In some embodiments, an ACE2 recombinant protein disclosed herein can be administered using a nebulizer capable of aerosolizing a unit dose at a preferred output rate. As used herein, a unit dose refers to a volume of the liquid pharmaceutical composition disclosed herein having the effective amount of the ACE2 recombinant protein designated to be administered during a single administration. In some embodiments, an ACE2 recombinant protein disclosed herein can be administered using a nebulizer capable of aerosolizing a unit dose at a rate of at least about 0.1 mL/minute to at least about 0.5 mL/minute. Assuming that the relative density of the liquid pharmaceutical composition herein is around 1, an ACE2 recombinant protein herein can be administered using a nebulizer capable of aerosolizing a unit dose at a rate of least about 100 mg/minute to about 500 mg/minute.

In some embodiments, an ACE2 recombinant protein disclosed herein administered using a nebulizer can remain stable through out dispersion into at least one of the subject's lungs. In some embodiments, an ACE2 recombinant protein disclosed herein administered using a nebulizer can retain dimerization through out dispersion into at least one of the subject's lungs. In some embodiments, at least 10% to at least 100% of the ACE2 recombinant protein dispersed into to at least one of the subject's lungs is a dimer. In some embodiments, an ACE2 recombinant protein herein administered using a nebulizer does not aggregate during dispersion into at least one of the subject's lungs. In some embodiments, at least about 10% to at least about 100% of the ACE2 recombinant protein dispersed into to at least one of the subject's lungs is not aggregated.

Targeted delivery of therapeutic compositions containing an antisense polynucleotide, expression vector, or subgenomic polynucleotides can also be used and is contemplated herein. Receptor-mediated DNA delivery techniques are described in, for example but not limited to, Findeis et al., Trends Biotechnol. (1993) 11:202; Chiou et al., Gene Therapeutics: Methods and Applications Of Direct Gene Transfer (J. A. Wolff, ed.) (1994); Wu et al., J. Biol. Chem. (1991) 266:338.

Therapeutic compositions containing a polynucleotide (e.g., those encoding the proteins described herein) can be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805: U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the methods described herein will depend on the particular subject and that subject's medical history.

In some embodiments, more than one ACE2 recombinant proteins, or a combination of an ACE2 recombinant protein and another suitable therapeutic agent, may be administered to a subject in need of the treatment. The ACE2 recombinant proteins disclosed herein can also be used in conjunction with other agents that serve to enhance and/or complement the effectiveness of the agents. Examples of therapeutic agents than can be administered prior to, after, or in combination with an ACE2 recombinant protein disclosed herein can be one or more treatments for a coronavirus infection. In some embodiments, the therapeutic agent used to treat a coronavirus infection can be one used to treat an infection caused by a virus that uses ACE2 as a co-receptor. In some embodiments, the therapeutic agent used to treat a coronavirus infection can be one used to treat a SARS-CoV-2 or a SARS-CoV-1 infection. In some embodiments, the therapeutic agent used to treat a coronavirus can be one given to a human subject having or suspected of having COVID-19. In some embodiments, ACE2 recombinant proteins disclosed herein can also be used in conjunction with one ore more antiviral drugs. Non-limiting examples of antiviral drugs for use herein can include remdesivir, favipiravir, merimepodib, lopinavir, ritonavir, and the like. In some embodiments, ACE2 recombinant proteins disclosed herein can also be used in conjunction with one or more corticosteroids. Non-limiting examples of corticosteroids for use herein can include prednisone, methylprednisolone, hydrocortisone, dexamethasone, and the like. Other examples of therapeutic agents that can be used in combination with any of the ACE2 recombinant proteins disclosed herein can include, but are not limited to anti-inflammatory drugs, convalescent plasma, amlodipine, ivermectin, losartan, famotidine, a monoclonal antibodies, polyclonal antibodies, antibiotics, and albuterol.

### IV. Kits

The present disclosure also provides kits for use in treating or alleviating a target disease, such as SARS infection (e.g., COVID-19) as described herein. Such kits can include one or more containers comprising an ACE2 recombinant protein, e.g., any of those described herein. In some instances, an ACE2 recombinant protein of the present disclosure may be co-used with a second therapeutic agent.

In some embodiments, kits disclosed herein can include one or more containers having at least one ACE2 recombinant protein disclosed herein and an aerosol delivery device. In some embodiments, an aerosol delivery device suitable for use here can be a nebulizer, a pressurized metered-dose inhaler (pMDI), or a dry powder inhaler (DPI).

In some embodiments, kits disclosed herein can include one or more containers having at least one ACE2 recombinant protein disclosed herein and a nebulizer delivery system. In some embodiments, a nebulizer delivery system can include at least a nebulizer and a source for compressed air. In some embodiments, a nebulizer delivery system can include tubing, mouthpiece, face mask, tent, an intermittent positive pressure breathing machine, or a combination thereof. In some embodiments, a nebulizer delivery system can include instructions for how to assemble the nebulizer, instructions for how to clean the nebulizer, instructions for how to care for the nebulizer, and the like.

In some embodiments, kits disclosed herein can include instructions for use in accordance with any of the methods described herein. The included instructions can comprise a description of administration of at least one ACE2 recombinant protein disclosed herein, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an ACE2 recombinant protein disclosed herein to an individual at risk of the target disease.

The instructions relating to the use of an ACE2 recombinant protein can generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert can indicate that the composition is used for inhibiting a coronavirus infection, inhibiting a SARS infection, treating COVID-19, or a combination thereof.

The kits disclosed herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an ACE recombinant protein as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### V. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Cabs, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»: Transcription and Translation (B. D. Hames & S. J. Higgins, eds. (1984»; Animal Cell Culture (R. I. Freshney, ed. (1986D; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. Design and Generation of ACE2 Constructs

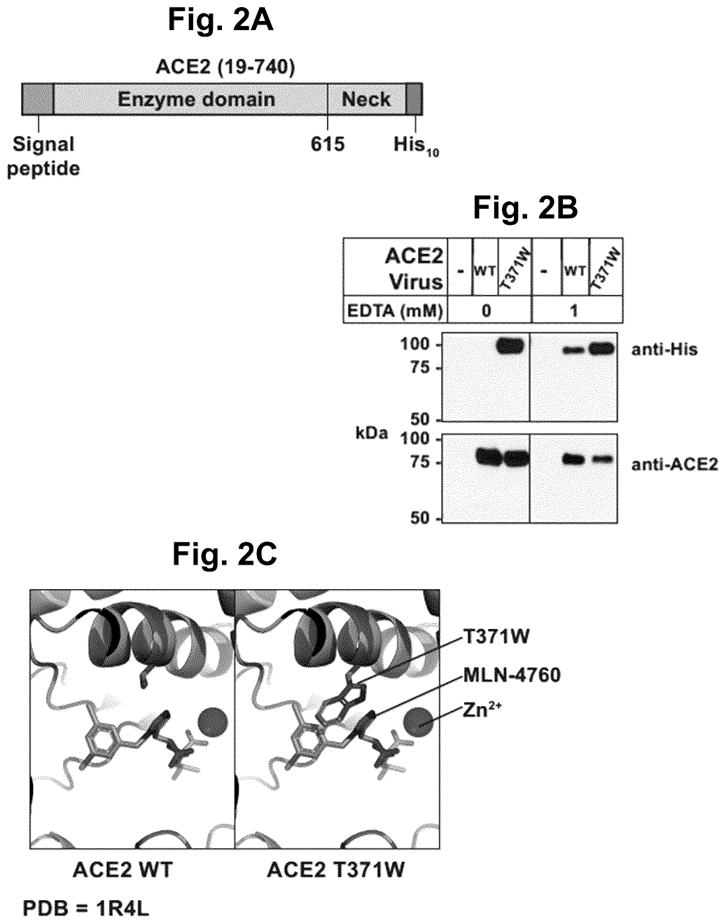

The native ACE2 ectodomain has two subdomains: an enzyme domain that can bind SARS2 and a neck domain which results in dimerization of the protein (FIG. 2A). The native ACE2 ectodomain has zincin carboxy-peptidase activity, which can cleave its C-terminus when expressed as soluble ectodomain. Experiments herein identify and develop human ACE2 recombinant proteins that lack protease activity and/or have improved protein characteristics such as, for example, protein stability.

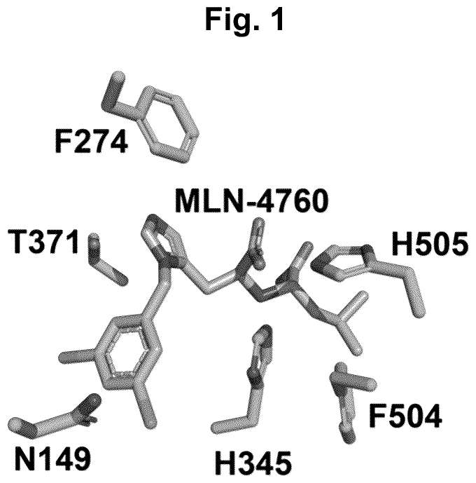

The human ACE2 amino acid sequence was subjected to computational and structural methods to identify potential residues and/or regions for mutation that could confer improved characteristics of human ACE2 recombinant proteins. Briefly, the ligand-bound crystal structure of ACE2 (PDB 1R4L) was used as a basis for the design of mutations which block ACE2 activity. Bound residues with side chains within 5 Å of the bound inhibitor (MLN-4760) were selected for examination to identify side chains which project into the ligand binding cavity but which do not have obvious structural function within the protein fold (FIG. 1). Importantly, residues that do not participate in the zinc chelation were selected, as disrupting the zinc molecule might destabilize the protein. The residues that met these criteria were: T371, N149, F274, H345, F504, H505.

Once promising residues and/or regions for mutation were identified in the human ACE2 protein sequence, plasmids were generated using standard molecular biology techniques. First, the DNA sequences of ACE2 coding regions as shown in Table 2 were prepared. Next, pFastBac-ACE2 plasmids were created using Gibson assembly methods to insert a synthetic dsDNA block having a SEQ ID NO: 11-18 into a pFastBac-1 plasmid that was linearized with BamHI and XhoI. The resulting constructs encoded a N-terminal Melittin signal peptide (MKFLVNVALVFMVVYISYIYA; SEQ ID NO: 19), the full-length human ACE2 ectodomain (residues 19-740; SEQ ID NO: 2), a TEV recognition site (ENLYFQG; SEQ ID NO: 20), a TG spacer, and/or 10 His residues (HHHHHHHHHH; SEQ ID NO: 21) forming a His tag at the C-terminal. FIG. 2A shows a schematic for the ACE2 ectodomain constructs. Using these methods, constructs were generated as described in Table 1 above.

FIG. 2C shows in particular, the design of the T371W mutation which prevents ACE2 protease activity. A crystal structure of the inhibitor-bound ACE2 enzymatic domain (PDB 1R4L) was used to identify residues which could be mutated to fill the binding pocket and prevent substrate engagement. Residue T371 projects into the binding pocket. The T371W mutation (SEQ ID NO: 10) inserts a large sidechain into the binding pocket and prevents proteolysis of the C-terminal His tag.

### Example 2. Expression and Purification of ACE2 Ectodomains

Plasmids from Example 1 were used to express recombinant ACE proteins using a baculovirus expression system. Briefly, baculoviruses were produced in Sf9 cells using the Bac-to-Bac Baculovirus Expression System. Sf9 cells at a density of 2 million cells/mL were infected with 20 mL of baculovirus per liter of culture and then cultured for 96 hours. Cells were harvested by centrifugation for 10 minutes at 6000×g at 4° C. and the supernatants were either used directly or else supplemented with 20% glycerol and frozen at −80° C. until purification.

To purify the ACE2 ectodomains, the supernatants were supplemented with protease inhibitors (160 μg/ml benzamidine, 100 μg/ml leupeptin, 1 mM PMSF, and 1 μM E-64), and further clarified by filtration with a 0.2 micron filter. Supernatants were concentrated and exchanged into an exchange buffer containing 50 mM Tris pH 8.0, 25 mM Imidazole pH 7.5, and 300 mM NaCl using a VIVAFLOW 200 (50 kDa MWCO) driven by a Masterflex L/S easy load peristaltic system. The concentrated and buffer-exchanged sample was incubated with NiNTA resin for 2-4 hours. The resin was washed with exchange buffer supplemented with Imidazole to final concentration of 50 mM. The ACE2 ectodomains were eluted in exchange buffer supplemented with Imidazole to final concentration of 300 mM. ACE2 ectodomains were then subjected to gel filtration in a superdex 200 column (GE) equilibrated in 20 mM HEPES pH 7.5 and 150 mM NaCl using an Akta Pure.

Sf9 cells uninfected or else infected with baculovirus for either ACE2 WT or ACE2 T371W were cultured with or without 1 mM EDTA supplemented in the culture media. Supernatants were harvested and immunoblotted with anti-His or anti-ACE2 antibodies to detect secreted ACE2 ectodomains. FIG. 2B shows that, while both proteins were expressed, WT ACE2 did not have a detectable His tag unless the enzyme was inhibited by EDTA.

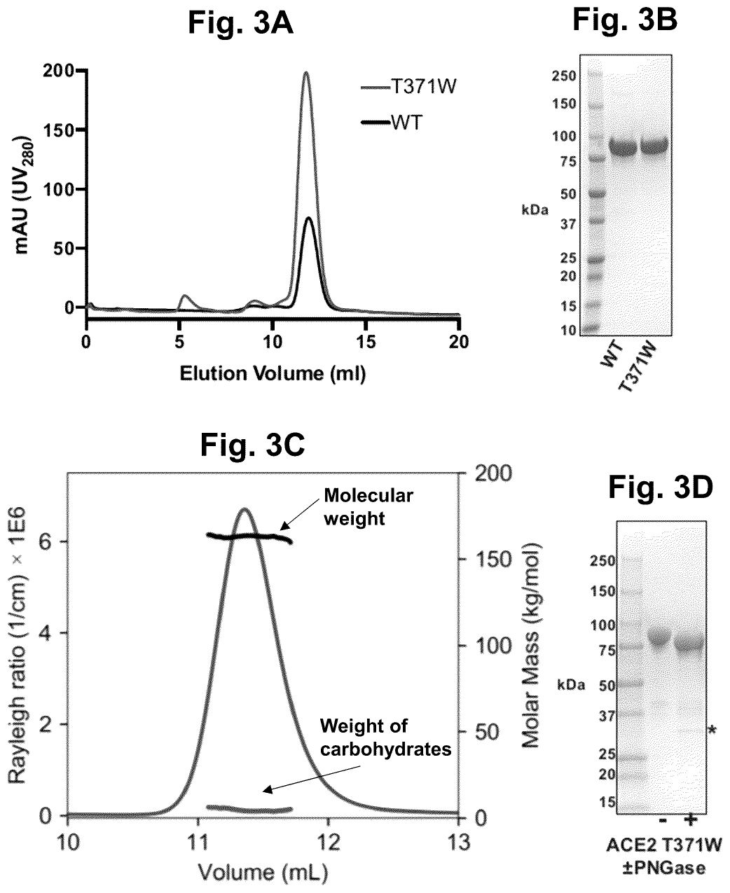

Purified ACE2 WT or ACE2 T371W ectodomains were subjected to superdex 200 gel filtration analysis as detailed above (FIG. 3A). The WT domain was expressed with 1 mM EDTA supplemented in the culture media. FIG. 3A shows that both forms of the protein had similar behavior. Purified ACE2 ectodomains were ran on a SDS-PAGE page gel which was stained with Coomassie blue (FIG. 3B).

To determine if the mutated ACE2 ectodomain was glycosylated, purified ACE2 T371W ectodomains were digested with PNGaseF prior to SDS-PAGE analysis. FIG. 3D shows that the ACE2 T371W ectodomain contained N-linked glycans as revealed by digestion with PNGaseF (“*” indicates PNGaseF band in FIG. 3D). Accordingly, the ectodomain ACE2 mutant ACE2 T371W was glycosylated.

Next, the purified ACE2 ectodomains, WT ACE2 and ACE2 T371W, were subjected to SEC-MALS analysis. Briefly, a Sephadex 200 Increase 10/300 size-exclusion column was equilibrated with gel filtration buffer. 400 μg of the sample at 2 mg/mL was injected and chromatographed at a flow rate of 0.5 mL/min. Data was recorded on a Shimadzu UV detector, a Wyatt TREOS II light-scattering detector, and a Wyatt Optilab t-REX differential refractive-index detector, which were all calibrated with a BSA standard.

A single peak eluted from the column after application of the ACE2 ectodomain. A protein conjugate analysis was performed using these data to account for the existence of both protein and carbohydrate in the form of glycosylation; the dn/dc value (i.e., refractive-index increment) for the protein was calculated based on its primary structure (0.1929 mL/g), and the assumed dn/dc for carbohydrate adducts was 0.15 mL/g. The resulting average molar mass of the protein component was 163,000±1,000 g/mol. There appeared to be only a small amount of the total mass associated with carbohydrate (4,600±1,100 g/mol). Accordingly, FIG. 3C shows that the purified ACE2 T371W ectodomain was a protein dimer in solution.

### Example 3. Stability Analysis of Purified ACE2 Ectodomains

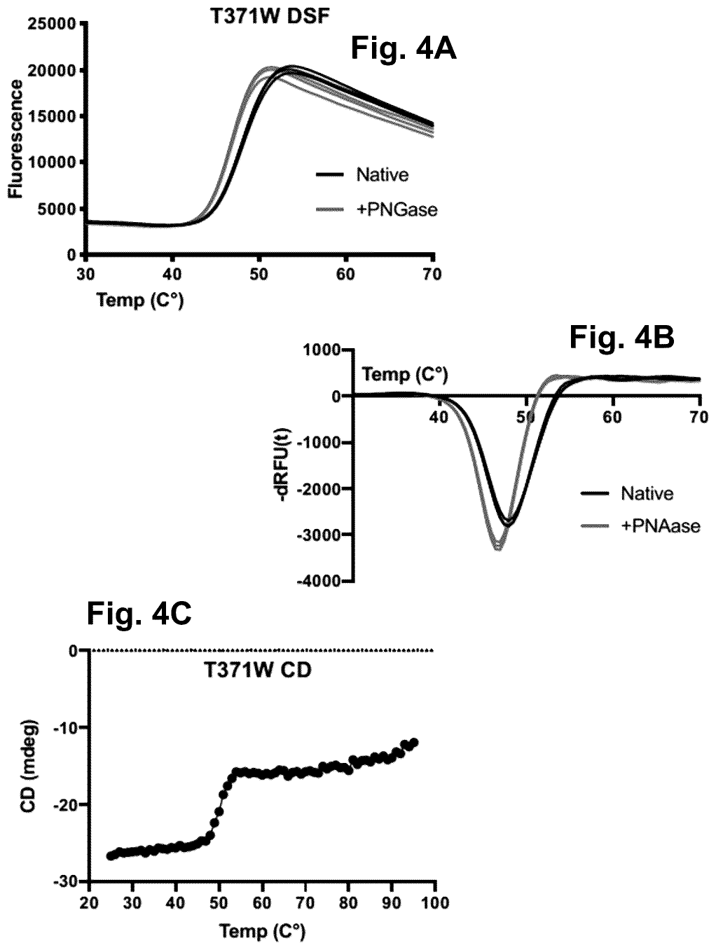

To assess thermal stability of the purified ACE2 T371W ectodomains, Differential Scanning Fluorimetry (DSF) experiments were performed. In brief, ACE2 T371W ectodomains were first either untreated or treated with PNGaseF. Next, the DSF experiments were conducted using a BioRad CFX96 Real-Time System machine. 20 μL reactions were set up with a protein concentration of 5 μM of ACE2 T371W ectodomains (untreated or treated with PNGaseF) in gel filtration buffer that was supplemented with SYPRO Orange dye at a final concentration of 5×. The temperature was increased from 20° C. to 95° C. at a rate of −1° C. per minute and fluorescence was recorded in all channels. The dye fluoresced most strongly in the HEX channel, and this data was used for the analysis. FIGS. 4A and 4B show that purified ACE2 T371W ectodomains had high thermal stability.

Circular dichroism (CD) spectroscopy was performed on MBR's Jasco J-815 CD Spectrometer. ACE2 T371W protein samples were prepared and placed in a 0.1 cm CD cuvette. The signal at 222 nm was plotted as a function of temperature, which was varied from 25° C. to 95° C. at a ramp rate of 2° C./minute. The signal revealed a transition at around 50° C. (FIG. 4C). Notably, the ACE2 T371W protein did not completely melt, as the CD signal persisted at the maximum experimental condition of 95° C. (FIG. 4C).

### Example 4. Binding of ACE2 Mutant Ectodomains to SARS2-RBD

Binding affinity and thermodynamics of the binding of the ACE2 T371W ectodomain to SARS-CoV-2 spike receptor-binding domain (SARS2-RBD) was measured by Isothermal Titration calorimetry (ITC). ITC is a quantitative technique that can determine the binding affinity (K_{D}), enthalpy changes (ΔH), and binding stoichiometry (η) of the interaction between two or more molecules in solution. From these initial measurements, Gibbs free energy changes (ΔG) and entropy changes (AS) can be determined using the relationship:

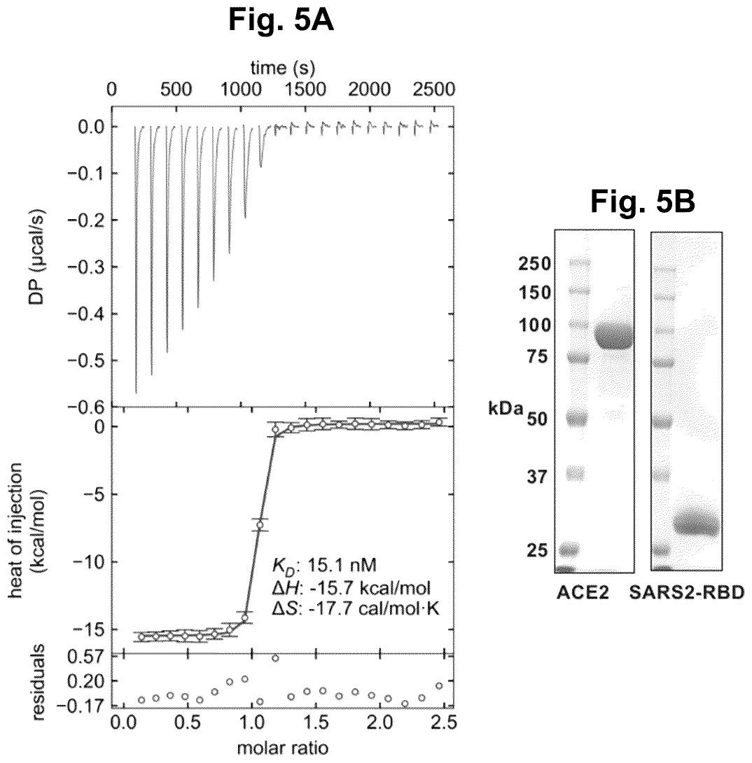

ΔG==RT ln K_{α}=ΔH−TΔS,

where R is the gas constant and T is the absolute temperature. Briefly, ITC experiments were conducted in a Malvern iTC200 calorimeter at 20° C. with protein samples in buffer containing 20 mM HEPES pH 7.5 and 150 mM NaCl. The calorimeter's stirred cell was filled with 209.5 μL ACE2 T371W at 20 μM concentration. The SARS2-RBD at a concentration of 200 μM was titrated into this cell. The first injection was 0.5 μL and then there were 20 injections of 1.9 μL. FIG. 5B shows a SDS-PAGE depicting the purity of the protein samples used in the ITC analysis.

The binding was determining to be a 1:1 interaction between the SARS2-RBD and ACE2 T371W monomers with a binding affinity (K_{D}) of 15.1 nM (95% confidence interval of 8-24 nM). The change in enthalpy (ΔH) was determined to be −15.7 kcal/mol (95% confidence interval −15.9-−15.4 kcal/mol). The change in entropy (AS) was determined to be −17.7 cal/mol·K. FIG. 5A.

### Example 5. Nebulization Assay of ACE2 Mutant Ectodomains

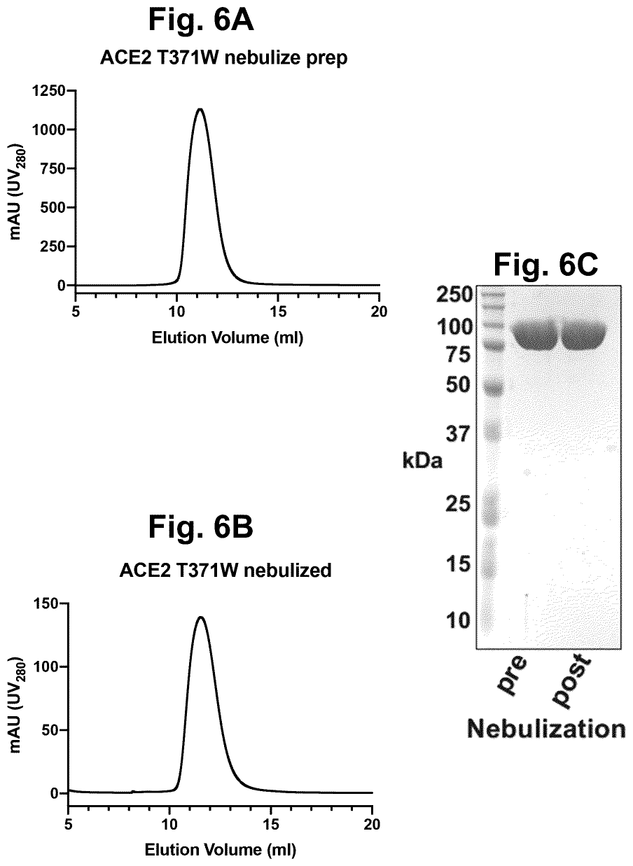

To determine if the ACE2 mutant ectodomains can tolerate nebulization, ACE2 T371W ectodomain was first purified in a buffer containing 20 mM HEPES pH 7.5 and 150 mM NaCl according to the methods described above. FIG. 6A shows elution of the purified ACE2 T371W ectodomain from Superdex S200 10/300 column.

To this sample, the excipient PEG 8000 was added to a final concentration of 1% w/v. The sample was then aerosolized by the vibrating mesh method using a NEB400 Mini Mesh Nebulizer. The aerosolized sample was collected and analyzed by Size Exclusion Chromatography over a Superdex S200 10/300 column. FIG. 6B shows the elution of the aerosolized ACE2 T371W ectodomain from the column.

FIGS. 6A and 6B show that the aerosolized ACE2 T371W ectodomain was free of aggregates and eluted as a homodimer at the same volume as the native protein. Also, there was little loss of the protein (93% of input concentration after nebulization). Samples of ACE2 T371W ectodomain were subjected to SDS-PAGE before and after nebulization. FIG. 5C shows that there was no change in the aerosolized ACE2 T371W ectodomain and non-aerosolized ACE2 T371W ectodomain.

### Example 6. Antiviral Screening Assay of ACE2 Mutant Ectodomains

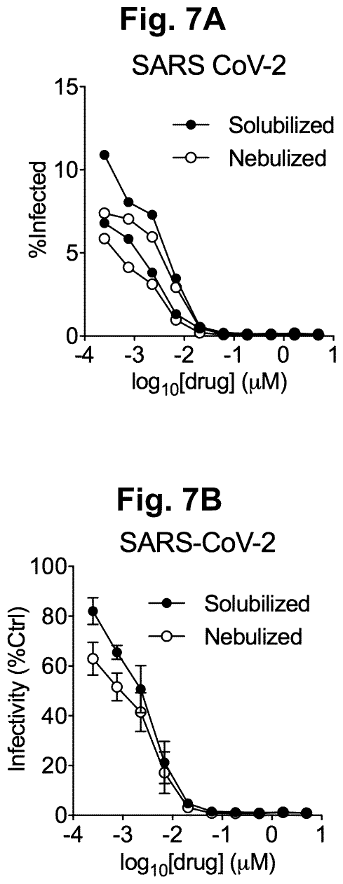

Next, the anti-SARS-CoV-2 activity of the ACE2 mutant ectodomain ACE2 T371W was assessed using an inhibition of infection assay. Briefly, on day 0, Vero E6 cells (a primate kidney cell line) were seeded in 24-well plates in at a density of 70,000 cells/well and grown overnight at 37° C. The next day, samples of soluble and nebulized ACE2 T1371W proteins were prepared. Both proteins were in nebulization buffer containing 20 mM HEPES pH 7.5, 150 mM NaCl, and 1% PEG 8000. Nebulization of the ACE2 T371W was conducted as described in Example 5. The “solubilized” ACE2 T371W was harvested before nebulization. The “nebulized” ACE2 T371W sample was harvested after nebulization. Serial dilutions of solubilized ACE2 T371W or nebulized ACE2 T371W were prepared in nebulization buffer at a final volume of 80 μL. These ACE2 T371W samples were mixed with 120 μL of serum-free Minimum Essential Media (MEM) containing either the SARS-CoV-2 virus or else a no-virus media control (mock) and incubated at 37° C. for 30 minutes. Subsequently, at time=0 hours, these samples were added to the Vero E6 cells, which were then incubated at 37° C. for 1 hour to permit infection of the Vero E6 cells by the SARS-CoV-2 virus. The final multiplicity of infection (MOI) by SARS-CoV-2 was ˜0.45 IU/cell. Following this incubation, the infection media was removed and replaced with 300 μL of MEM culture medium supplemented with 10% serum. At time=7.5 hours, the cells were harvested in 150 uL Accumax and then the virus was inactivated by adding paraformaldehyde to a final concentration of 4%. Cells were subjected to flow cytometry where infectivity was determined by antibody stain for viral N protein. Data were normalized to the mock-infected control, after which EC5 0 values were calculated. Data show that both the solubilized ACE2 T371W and the nebulized ACE2 T371W had an EC₅₀ of about 1-2 nM (FIGS. 7A and 7B).

### Example 7. Viral Entry Analysis of Purified ACE2 Ectodomains

The viral entry blocking effect of purified ACE2 T371W ectodomains is determined. By one method, a SARS-CoV-2 Spike protein expressing pseudotyped lentivirus is used to infect cells and, in turn, estimate the lentiviral titer. Preincubating the pseudotyped lentivirus with cells allows for assessment of the ACE2 T371W ectodomains' ability to block viral entry. In another method, the viral entry blocking effect of ACE2 T1371W ectodomains is confirmed using SARS-CoV-2 isolated from patients suffering from COVID-19 and performing a plaque reduction assay with the isolated virus and ACE2 T371W ectodomains.

### Example 8. In Vivo Infections of a Mouse Model with SARS-CoV-2

Mouse models of SARS-CoV-2 were used to assess effectiveness of in vivo administration of a recombinant ACE protein of the present disclosure. In brief, female K18-hACE c57BL/6J mice (strain: 2B6.Cg-Tg(K18-ACE2)₂Prlmn/J; Jackson Laboratory) aged 6-8 weeks were anesthetized with ketamine/xylazine (80/6 mg/kg) and intranasally (IN) infected with 1×10² PFU of SARS-CoV-2 P.1 variant suspended in 30 μl of phosphate-buffered saline (PBS). The mice were either untreated or treated 20 μl IN with stabilized ACE2 T371W (sACE2; 11.6-12 mg/ml) 30 minutes either before or after infection, or were given sACE2 1 day post infection, 2 day post infection, or combination of 1 and 2 days post infection. All mice were monitored daily for weight and mortality. Animals that lost more than 20% of their original body weight or appear lethargic, hunched, or unable to obtain food were euthanized.

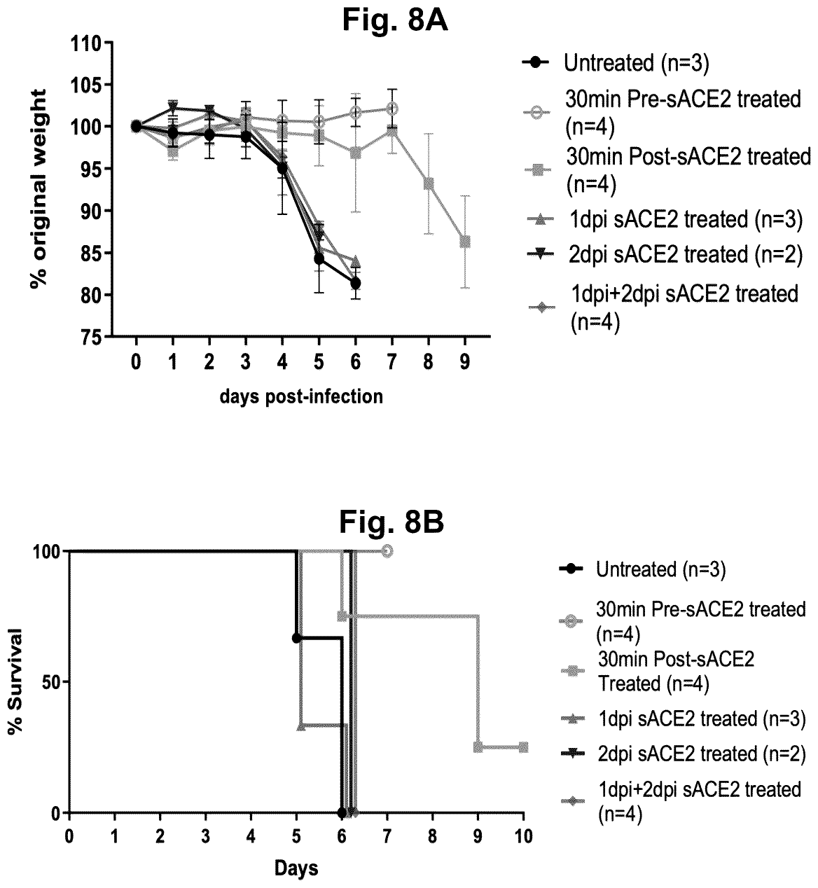

As shown in FIG. 8A, mice treated with sACE2 30 minutes before and after infection with the SARS-CoV-2 P.1 variant were protected from weight loss for up to 7 days post-infection. FIG. 8B shows that SARS-CoV-2-infected mice pre-treated or post-treated with sACE2 were protected from death compared to control, untreated infected mice. Together, these data showed that intranasal administration of sACE2—a stabilized ACE2 ectodomain—increased probability of survival and was protective against death resulting from SARS-CoV-2 infection.

### Example 9. Site-specific PEG-modified Versions of Mutant ACE2 Ectodomains

Structural analysis of the ACE2 ectodomain indicated that all endogenous cysteines were either engaged in disulfide bonds or are buried within the structure such that they would not be reactive with a soluble reactive compound. Accordingly, introduction of a free cysteine residue on the surface of the protein faced away from the coronavirus binding site, which was labeled with a polyethylene glycol (PEG) moiety in a specific manner. A PEG-modified version of the ACE2 ectodomain proteins described herein provided improved pharmacokinetics and immunoreactivity profiles relative to the unmodified protein, as has been described for other protein therapeutics.

Cysteine residues were incorporated into ACE2 ectodomain polypeptides already mutated to enhance stability (e.g. T371W). Cysteine mutations were targeted to positions that are predicted to be solvent-accessible, such as at or near the ectodomain C-terminus. Examples of ACE2 ectodomain sequences that were been modified in this manner are: ACE2 T371W/G732C and ACE2 T371W/S740C, the amino acid sequences and polynucleotide sequences of which are provided in Table 3 herein.

ACE2 ectodomain constructs modified to express ACE2 ectodomains with a surface-accessible cysteine residue are expressed and purified as described above for the other ACE2 constructs. Following the final purification step where the protein is purified by gel filtration in a suitable buffer such as phosphate buffered saline (PBS) or a buffer consisting of 20 mM HEPES pH 7.5 and 150 mM NaCl, the protein is modified by conjugation with a cysteine-reactive chemical such as one containing a Maleimide or bromine moiety, which reacts with the exposed thiol on the introduced cysteine residue. In a typical procedure, the purified protein is incubated with a 3, 5, or 10-fold molar excess of maleimide-PEG reagents for 30 minutes at 25° C. The modified protein is separated from unreacted compounds by gel filtration in a similar buffer. The cysteine modification is confirmed by analytical techniques including mass spectrometry or SDS-PAGE methods to assess the percentage of protein which has been modified. After optimization of the reaction to achieve stoichiometric labeling of the introduced cysteine, the PEG-modified ACE2 ectodomain samples are used in in-vitro or in-vivo assays as described herein.

1. A recombinant protein, comprising a fragment of an angiotensin-converting enzyme 2 (ACE2) receptor, the fragment having at least 80% identity to an amino acid sequence represented by SEQ ID NOs: 3-9 or a nucleic acid encoding the fragment of an ACE2 receptor comprising at least 80% identity with the polynucleotide represented by SEQ ID NOs: 11-17, wherein the fragment comprises at least one binding site for a spike protein of a coronavirus.

2. The recombinant protein of claim 1, wherein the ACE2 receptor is a human ACE2 receptor.

3. The recombinant protein of claim 1, wherein the fragment of the ACE2 receptor comprises the ectodomain of the ACE2 receptor.

4. The recombinant protein of claim 1, which further comprises a signaling peptide at the N-terminus.

5. The recombinant protein of claim 4, comprising a polypeptide having at least 90% identity to the amino acid sequence represented by SEQ ID NOs: 3-9.

6. The recombinant protein of claim 5, comprising a polynucleotide sequence encoding a fragment of an ACE2 receptor having at least 90% identity to SEQ ID NOs: 11-17.

7. The recombinant protein of claim 5, which comprises the amino acid sequence of SEQ ID NO: 10.

8. The recombinant protein of claim 6, which comprises the polynucleotide sequence of SEQ ID NO: 18.

9. The recombinant protein of claim 1, wherein the protein is PEGylated.

10. The recombinant protein of claim 9, wherein the PEGylated protein comprises a polypeptide sequence having at least 90% identity to SEQ ID NOs: 22-23.

11. The recombinant protein of claim 9, wherein the PEGylated protein comprises a polynucleotide sequence encoding a fragment of an ACE2 receptor having at least 90% identity to SEQ ID NOs: 24-25.

12. The recombinant protein of claim 1, wherein the coronavirus is SARS-CoV-2.

13. A pharmaceutical composition, comprising a recombinant protein set forth in claim 1 and a pharmaceutically acceptable carrier.

14. The pharmaceutical composition of claim 13, wherein the pharmaceutical composition is a nebulized solution.

15. A method of treating a coronavirus infection, the method comprising administering to a subject in need thereof an effective amount of the recombinant protein of claim 1.

16. The method of claim 15, wherein the subject is a human subject having, suspected of having, or at risk for the coronavirus infection.

17. The method of claim 16, wherein the coronavirus infection is SARS-CoV-2 infection.

18. The method of claim 17, wherein the subject is a human patient having or suspected of having COVID-19.

19. The method of claim 15, further comprising administering to the subject an effective amount of least one anti-viral agent.

20. The method of a claim 19, wherein the least one anti-viral agent is remdesivir.

21. The method of claim 15, further comprising administering to the subject an effective amount of at least one corticosteroid.

22. The method of a claim 21, wherein the least one corticosteroid is dexamethasone.

23. The method of claim 15, wherein the recombinant protein is administered by a nebulizer.


SEQUENCE LISTING (25 sequences)

SEQ ID NO: 1 - protein, 805 aa, Homo sapiens [sequence omitted]

SEQ ID NO: 2 - protein, 598 aa, Homo sapiens
  Ectodomain of Human ACE2
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 762 aa, Artificial Sequence
  synthetic sequence; WT ACE2
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 4 - protein, 762 aa, Artificial Sequence
  synthetic sequence; ACE2 T371X
  misc_feature (374)..(374): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLXAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 5 - protein, 762 aa, Artificial Sequence
  synthetic sequence; N149X ACE2
  misc_feature (152)..(152): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLXEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 6 - protein, 762 aa, Artificial Sequence
  synthetic sequence; F274X ACE2
  misc_feature (277)..(277): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRXWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 7 - protein, 762 aa, Artificial Sequence
  synthetic sequence; H345X ACE2
  misc_feature (348)..(348): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCXPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 8 - protein, 762 aa, Artificial Sequence
  synthetic sequence; F504X ACE2
  misc_feature (507)..(507): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLXHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 9 - protein, 762 aa, Artificial Sequence
  synthetic sequence; H505X ACE2
  misc_feature (508)..(508): Xaa can be any naturally occurring amino acid
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFXVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 10 - protein, 762 aa, Artificial Sequence
  synthetic sequence; ACE2 T371W
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLWAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSENLYFQGTGHHHHHHHHHH

SEQ ID NO: 11 - DNA, 2289 bp, Artificial Sequence
  synthetic sequence; WT ACE2
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 12 - DNA, 2289 bp, Artificial Sequence
  synthetic sequence; ACE2 T371X
  misc_feature (1120)..(1122): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgnnngcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 13 - DNA, 2289 bp, Artificial Sequence
  synthetic sequence; ACE2 N149X
  misc_feature (454)..(456): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcnnngaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 14 - DNA, 2289 bp, Artificial Sequence
  synthetic sequence; ACE2 F274X
  misc_feature (829)..(831): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcnnntggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 15 - DNA, 2289 bp, Artificial Sequence
  synthetic sequence; ACE2 H345X
  misc_feature (1042)..(1044): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgcnnncccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 16 - DNA, 2289 bp, Artificial Sequence
  synthetic sequence;  ACE2 F504X
  misc_feature (1519)..(1521): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgnnncatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 17 - DNA, 2289 bp, Artificial Sequence
  synthetic sequence; ACE2 H505X
  misc_feature (1522)..(1524): n is a, c, g, t or u
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgactgcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttnnngtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtcggagaatctctacttccagggtaccggtcatcaccaccaccaccatcaccatcatcattga

SEQ ID NO: 18 - DNA, 2223 bp, Artificial Sequence
  synthetic sequence; ACE2 T371W
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgtgggcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccg

SEQ ID NO: 19 - protein, 21 aa, Artificial Sequence
  synthetic sequence; Melittin signal peptide
MKFLVNVALVFMVVYISYIYA

SEQ ID NO: 20 - protein, 7 aa, Artificial Sequence
  synthetic sequence; TEV recognition site
ENLYFQG

SEQ ID NO: 21 - protein, 10 aa, Artificial Sequence
  synthetic sequence; His tag
HHHHHHHHHH

SEQ ID NO: 22 - protein, 762 aa, Artificial Sequence
  synthetic sequence; ACE2 T371W/G732C
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLWAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLCPPNQPPVSENLYFQGTGHHHHHHHHHH

SEQ ID NO: 23 - protein, 762 aa, Artificial Sequence
  synthetic sequence; ACE2 T371W/S740C
MKFLVNVALVFMVVYISYIYASTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLWAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVCENLYFQGTGHHHHHHHHHH

SEQ ID NO: 24 - DNA, 2289 bp, Artificial Sequence
  synthetic sequence; ACE2 T371W/G732C
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgtgggcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgtgccctccaaaccagcctccggtgtcggagaatctctacttccagggtaccggtcatcaccaccaccaccatcaccatcatcattga

SEQ ID NO: 25 - DNA, 2289 bp, Artificial Sequence
  synthetic sequence; ACE2 T371W/S740C
atgaaatttctggtcaatgtagccctggtattcatggtcgtctacatctcgtatatatatgcctcgacaatagaagaacaagctaagacttttctcgacaaatttaaccatgaagcagaggacctcttctaccagtcatcgttggcatcgtggaactacaacacgaatataaccgaggaaaacgttcagaatatgaacaacgccggcgataagtggagcgccttccttaaggagcaatctacgctggcccagatgtatcccttgcaggagattcaaaatcttacggttaagcttcagctgcaggccctgcagcaaaatggttcaagcgtactcagtgaagataaatctaagagactgaatacaattctcaacaccatgagcaccatttactctacaggaaaggtgtgcaaccccgataatccacaagagtgcctgttgctggagcctggcctcaatgaaattatggcaaacagtcttgactacaatgagaggctctgggcgtgggagtcttggaggagtgaggtgggaaagcagctgcgcccactgtatgaagagtatgtagtccttaagaacgagatggcgcgcgcaaatcattatgaggattatggagattactggcgtggcgactatgaagtgaatggcgtcgacggatacgattattcgcgcggccaactcatcgaggatgtagagcacactttcgaagagataaagcctttgtacgagcaccttcacgcgtacgttagggccaagcttatgaatgcttacccatcttatattagccctatcggctgcctgcctgcgcatctgttgggagatatgtggggtcgcttctggaccaacctctattctcttactgtcccattcggccaaaagccaaatatcgacgtgacagatgccatggtcgatcaagcatgggacgcccaaaggatattcaaggaagccgaaaaattttttgttagtgtcggcctccccaacatgacgcaaggtttctgggagaattctatgctcacggatccaggtaacgtgcagaaggccgtatgccaccccacagcctgggacttgggtaaaggagacttcaggattttgatgtgtaccaaagttaccatggatgattttctgtgggcacaccacgaaatgggccacattcagtatgatatggcttacgctgctcaaccatttcttcttcgcaatggagccaacgagggatttcatgaagcagtaggtgaaataatgtcgctgagtgcggcaacaccgaagcaccttaagtccattggactgctcagccccgactttcaggaagataacgagacggagataaacttcctgctcaagcaggcactcacgatcgtgggaacgttgccttttacgtatatgcttgaaaagtggagatggatggtgttcaaaggcgaaatacctaaagaccagtggatgaaaaagtggtgggaaatgaagagagaaatagtcggagtcgtcgaacccgtgccgcatgatgaaacctactgcgatcctgccagcttgtttcatgtgagtaatgattatagtttcatacgctactatactcgcacgttgtaccagtttcaatttcaggaggccctgtgtcaagcggcaaagcatgagggaccgctgcacaagtgcgacataagtaatagcactgaggctggacaaaagctgtttaacatgctgcgtctcggaaagagtgagccttggactctcgcactggaaaatgtcgttggcgcgaaaaacatgaatgtacgtccgcttctgaattactttgagccattgtttacatggttgaaagatcagaataaaaatagttttgtgggctggtcaacggattggagcccttacgctgatcaatctataaaggtgagaatatcccttaagagtgcgttgggagataaggcatacgagtggaacgacaatgaaatgtacttgttccgctcttcagtagcatacgctatgaggcagtatttccttaaagtaaaaaatcagatgatcttgtttggcgaagaagatgtccgcgttgctaaccttaagccaagaatttcgttcaacttttttgttaccgcaccaaagaatgtctcagacatcataccccgcactgaagtagaaaaagcgattagaatgtcgcgtagtcgtatcaacgacgcgtttaggctcaacgacaattcgctggaattcctgggaattcagcccacattgggccctccaaaccagcctccggtgtgcgagaatctctacttccagggtaccggtcatcaccaccaccaccatcaccatcatcattga